(19) United States
(12) Patent Application Publication
(10) Pub. No.: US 2022/0148677 A1
(43) Pub. Date: May 12, 2022

(54) METHODS AND SYSTEMS FOR DETECTING GENETIC FUSIONS TO IDENTIFY A LUNG DISORDER

(71) Applicant: Veracyte, Inc., South San Francisco, CA (US)

(72) Inventors: Giulia C. Kennedy, San Francisco, CA (US); Patric Sean Walsh, South San Francisco, CA (US); Yangyang Hao, South San Francisco, CA (US); Jing Huang, South San Francisco, CA (US); Joshua Babiarz, South San Francisco, CA (US)

(21) Appl. No.: 17/349,830

(22) Filed: Jun. 16, 2021

Related U.S. Application Data
(63) Continuation of application No. PCT/US2019/067975, filed on Dec. 20, 2019.
(60) Provisional application No. 62/861,752, filed on Jun. 14, 2019.
(60) Provisional application No. 62/782,819, filed on Dec. 20, 2018.

(51) Int. Cl. G16B 20/20; G16B 40/20; C12Q 1/6886; G16H 10/40; G16H 50/20; G16H 15/00

(52) U.S. Cl. CPC G16B 20/20; G16B 40/20; C12Q 1/6886; C12Q 2600/112; G16H 50/20; G16H 15/00; C12Q 2600/158; G16H 10/40

(57) ABSTRACT

Gene fusions are hybrid genes formed by two previously separate genes. Many such gene fusions may be strong driver mutations for cancer and may play important roles in tumorigenesis. Gene fusions may be identified in biological samples extracted from the diseased location. However, for lung cancer, extracting samples from the nodules may be challenging and may lead to undesirable consequences. Disclosed herein is a method and system of identifying a gene fusion landscape from bronchial brushings and further identifying fusions that may be potentially associated with lung cancer.

### CROSS-REFERENCE

This application is a continuation application of International Patent Application No. PCT/US2019/067975, filed Dec. 20, 2019, which claims priority to U.S. Provisional Application No. 62/861,752, filed Jun. 14, 2019 and U.S. Provisional Application No. 62/782,819, filed Dec. 20, 2018, each of which is entirely incorporated herein by reference.

### BACKGROUND

There are various types of lung conditions, such as diseases that may affect the lungs or airways of subjects. Examples of lung diseases include but are not limited to lung cancer, COPD, cystic fibrosis, chronic bronchitis, asthma, pneumonia, pulmonary edema, and pseumoconiosis.

Lung cancer is a type of cancer that may be due to abnormal tissue grown in a lung of a subject. Lung cancer may have a genetic basis (e.g., the subject is genetically predisposed to abnormal cell growth in the lungs of the subject), environmental basis (e.g., exposure to pollutants, such as cigarette smoke), or both.

Methods currently available for detecting lung conditions, such as lung cancer, may not be able to (i) to assess a subject's risk for developing a lung condition or (ii) to detect many lung conditions in their early stages. Additionally, such methods may involve highly invasive and painful procedures.

### SUMMARY

The present disclosure provides methods and systems for detecting a lung condition in a subject, such as a lung disease (or disorder). Such lung disease may be lung cancer. Methods of the present disclosure may include detecting lung cancer associated fusions in bronchial brushing samples.

In an aspect, the present disclosure provides a method for processing or analyzing a sample of epithelial tissue from a respiratory tract of a subject, comprising providing the sample of epithelial tissue from a respiratory tract of the subject, wherein the sample comprises gene expression products; assaying the gene expression products of the sample by sequencing, sequence hybridization, array hybridization, or nucleic acid amplification to yield data; in a programmed computer, using said data to determine a presence or absence of one or more gene fusions; and electronically outputting a report that identifies a classification of the sample of epithelial tissue from the respiratory tract of the subject as positive or negative for the lung cancer. The method can further comprise obtaining the sample by a bronchoscopy. The method can further comprise obtaining the sample by fine needle aspiration. The sample can be a bronchial brushing sample. The method can further comprise obtaining the sample by bronchial brushing. Said subject can have lung nodules that are inconclusive for lung cancer, as determined by computed tomography (CT) scan or bronchoscopy. The sample can be inconclusive for lung cancer. The sample can comprise a mucous epithelial tissue, a nasal epithelial tissue, a lung epithelial tissue, or any combination thereof. The sample can comprise epithelial tissue obtained along an airway of the subject. The gene expression products can be ribonucleic acid. The gene expression products can be deoxyribonucleic acid. The nucleic acid amplification can comprise contacting at least one target sequence within the gene expression products with a nucleic acid probe under conditions wherein the probe forms hybridization complexes with the at least one target sequence, wherein the probe comprises the target specific sequence and an adapter sequence that is unique to the gene expression products.

The method can further comprise modifying the probe that forms hybridization complexes, thereby forming a modified probe. The method can further comprise detecting the presence of the adapter sequence in the modified probe, thereby identifying the at least one target sequence in the sample.

Detecting can further comprise contacting the modified probe with a nucleic acid array comprising sequences complementary to the adapter sequence. Detecting can further comprise contacting the modified probe with a solid support comprising sequences complementary to the adapter sequence. The solid support can be a bead. Detecting can further comprise amplifying the modified probe. Using the data to determine the presence or absence of one or more gene fusions can further comprise using a trained algorithm wherein the trained algorithm is trained by a training data set.

The training data set can comprise data from samples benign for a lung condition and samples malignant for the lung condition. The training data set can comprise data from samples obtained from subjects associated with the risk of developing lung cancer. The risk of developing lung cancer can include high risk, intermediate risk, and low risk. The high risk can be greater than 60%. The low risk can be less than 10%. The training data set can comprise bronchial brushing samples obtained before and/or after collection of a clinical sample. The training data set can comprise samples obtained from subjects using inhaled medication.

Using said data to determine a presence or absence of one or more gene fusions can further comprise using a trained algorithm that uses the data to determine the presence or absence of one or more gene fusions. The trained algorithm can be trained by a training set. The training data set can comprise data from samples benign for a lung condition and samples malignant for a lung condition. The training data set can comprise data from samples obtained from subjects associated with the risk of developing lung cancer. The trained algorithm can comprise a covariate. The covariate can be a self-reported characteristic. The self-reported characteristic can be exposure to an inhaled medication and the covariate can be a weight applied to gene expression data of genes associated with exposure to an inhaled medication. The trained algorithm can comprise a first filter and a second filter wherein the first filter identifies gene fusion candidates and the second filter identifies refined gene fusion candidates to generate the classification. The method can further comprise the first filter filtering the data by the number of junction reads. The number of junction reads can be greater than three. The number of junction reads can be equal to three.

The method can further comprise the second filter identifying refined gene fusion candidates based off scoring in the (i) a prevalence value of a gene fusion in both bronchial brushing lung cancer and TBB benign patient cohorts; (ii) a risk ration (RR); and (iii) a positive predicative value (PPV). The prevalence value can comprise a detection number and a detection percent. The detection number can be a number of the samples in which the gene fusion was detected. The detection percent can be a percent of frequency of gene fusion detection in non-lung cancer samples. The method can further comprise calculating the risk ratio from the following formula:

$$R R = \frac{{M}_{f} / {N}_{f}}{{M}_{n o f} / {N}_{n o f}}$$

wherein M_{f} is the number of malignant cancer samples in which the gene fusion is detected, M_{nof} is the number of malignant cancer samples in which the gene fusion is not detected, B_{f} is the number of benign cancer samples in which the gene fusion is detected, B_{nof} is the number of malignant cancer samples in which the gene fusion not detected, N_{f} is the sum of M_{f} and B_{f}, and N_{nof} is the sum of M_{nof} and B_{nof}. The method can further comprise calculating the positive predictive value from the following formula:

$$P P V = \frac{N ( {M}_{f} )}{N ( {N}_{f} )}$$

wherein N(M_{f}) is a function of M_{f} and N(N_{f}) is a function of N_{f}, The method can further comprise the second filter requiring a positive predicative value >0.5. The method can further comprise the second filter requiring a risk ratio >1. The gene fusion can be considered to be associated with lung cancer if the risk ratio is >1 among all samples and if a gene fusion was detected at least once in Primary-risk ratio >1. A gene fusion can be considered to be associated with lung cancer if the positive predicative value is >0.5 among all samples.

The one or more gene fusions can be selected from ASCC2_DEK, EVPL_HLA-DRB5, BPIFB1_FAM73B, ARID4B_TMX4, TFDP2_XRN1, H6PD_SPSB1, APP_GTF3C1, PPL_SAA1, LRP10_MUC5AC, C1orf87_CD36, HLA-DRB5_ZNF497, IKZF5_MUC5AC, EPPK1_LHCGR, C19orf33_MRPL30, EHD3_KIAA1429, MYO9B_WASF2, GGNBP2_MYO19, CCDC64B_CCDC78, POLR1A_REEP1, ATXN3_MAML2, CPSF6_FAM203B, MUC16_MUC4, OS9_RYR1, PPFIA3_TRPM4. The data can correspond to levels of the one or more gene expression products.

The identification of the sample of epithelial tissue from the respiratory tract of the subject as positive for lung cancer can comprise identifying the sample as high risk for lung cancer. The identification of the sample of epithelial tissue from the respiratory tract of the subject as negative for lung cancer can comprise identifying the sample as low risk for lung cancer. The sample can be inconclusive for lung cancer. Assaying can further comprise electronically outputting a report that identifies a classification of the sample as positive for lung cancer. An output of positive for lung cancer may indicate that the lung cancer is a malignant lung cancer. An output of positive for lung cancer may indicate that the lung cancer is a benign lung cancer. Assaying can comprise electronically outputting a report that identifies a classification of said sample as negative for lung cancer.

In an aspect, the present disclosure provides a system comprising a communication interface that is configured to receive, over a communication network, data derived from assaying gene expression products of a sample, one or more computer processors in communication with said communication interface, wherein the one or more computer processors are individually or collectively programmed to implement a method comprising: (i) receiving, over the communication network, the data derived from assaying gene expression products of the sample, (ii) detecting the level of one or more gene fusions in the sample, to yield gene fusion data corresponding to the level(s) of said one or more gene fusions; (iii) inputting said gene fusion data into a programmed computer to classify the sample as positive or negative for lung cancer; and (iv) electronically outputting an electronic report comprising said classification of (iii) that identifies said classification of said sample as positive or negative for said lung cancer. The system can further comprise an assay unit that is configured to assay the gene expression products of the sample by sequencing, sequence hybridization, array hybridization, or nucleic acid amplification. The one or more gene fusions can be association with a risk of developing lung cancer.

The programmed computer can use a trained classifier trained by a training data set. The training data set can comprise samples benign for a lung condition and samples malignant for lung cancer. The training data set can comprise samples obtained from subjects associated with a risk of developing lung cancer. The trained algorithm can comprise a first filter and a second filter wherein the first filter identifies gene fusion candidates and the second filter identifies refined gene fusion candidates to generate said classification. The first filter can filter the data by the number of junction reads. The second filter can identify refined gene fusion candidates based off scoring in (i) a prevalence value of a gene fusion in both bronchial brushing cancer and transbronchial biopsy (TBB) benign patient cohorts; (ii) a risk ratio (RR); and (iii) a positive predicative value (PPV). The prevalence value can comprise a detection number and a detection percent. The detection number can be a number of the samples in which the gene fusion was detected. The detection percent can be a percent of frequency of gene fusion detection in non-lung cancer samples. The risk ratio can be calculated from the following formula:

$$R R = \frac{{M}_{f} / {N}_{f}}{{M}_{n o f} / {N}_{n o f}}$$

wherein M_{f} is the number of malignant cancer samples in which the gene fusion is detected, M_{nof} is the number of malignant cancer samples in which the gene fusion is not detected, B_{f} is the number of benign cancer samples in which the gene fusion is detected, B_{nof} is the number of malignant cancer samples in which the gene fusion not detected, N_{f} is the sum of M_{f} and B_{f}, and N_{nof} is the sum of M_{nof} and B_{nof}. The positive predicative value can be calculated from the following formula:

$$P P V = \frac{N ( {M}_{f} )}{N ( {N}_{f} )}$$

wherein N(M_{f}) is a function of M_{f} and N(N_{f}) is a function of N_{f}, The second filter can require a positive predicative value >0.5. The second filter can require a risk ratio >1. The gene fusion can be considered to be associated with lung cancer if the risk ratio is >1 among all samples and if a gene fusion was detected at least once in Primary-risk ratio >1. A gene fusion can be considered to be associated with lung cancer if the positive predicative value is >0.5 among all samples.

The one or more gene fusions can be selected from ASCC2_DEK, EVPL_HLA-DRB5, BPIFB1_FAM73B, ARID4B_TMX4, TFDP2_XRN1, H6PD_SPSB1, APP_GTF3C1, PPL_SAA1, LRP10_MUC5AC, C1orf87_CD36, HLA-DRB5_ZNF497, IKZF5_MUC5AC, EPPK1_LHCGR, C19orf33_MRPL30, EHD3_KIAA1429, MYO9B_WASF2, GGNBP2_MYO19, CCDC64B_CCDC78, POLR1A_REEP1, ATXN3_MAML2, CPSF6_FAM203B, MUC16_MUC4, OS9_RYR1, PPFIA3_TRPM4. The data can correspond to levels of the one or more gene expression products.

The identification of the sample of epithelial tissue from the respiratory tract of the subject as positive for lung cancer can comprise identifying the sample as high risk for lung cancer. The identification of the sample of epithelial tissue from the respiratory tract of the subject as negative for lung cancer can comprise identifying the sample as low risk for lung cancer. The sample can be inconclusive for lung cancer. Assaying can further comprise electronically outputting a report that identifies a classification of the sample as positive for lung cancer. An output of positive for lung cancer may indicate that the lung cancer is a malignant lung cancer. An output of positive for lung cancer may indicate that the lung cancer is a benign lung cancer. Assaying can comprise electronically outputting a report that identifies a classification of said sample as negative for lung cancer.

In an aspect, the present disclosure provides a system for processing or analyzing a sample of epithelial tissue of a subject. The system may comprise a sequencing unit configured to process a sample comprising gene expression products to generate data, a trained classifier algorithm, and an electronic output unit. The data may be derived from sequencing, array hybridization, or nucleic acid amplification corresponding to a presence or absence of one or more gene fusions wherein one or more gene fusions are associated with a risk of developing lung cancer. The trained classifier algorithm may be configured to generate a classification of the sample of tissue as positive or negative for the lung cancer. The electronic output may identify the classification of the sample of tissue as positive or negative for lung cancer. The sample may be obtained by bronchoscopy. The sample may be obtained by fine needs aspiration. The sample may comprise a mucous epithelial tissue, a nasal epithelial tissue, a lung epithelial tissue, or any combination thereof. The sample may comprise an epithelial tissue obtained along an airway of the subject.

In another aspect, the present disclosure provides a method for processing or analyzing a sample of epithelial tissue of a subject comprising (i) obtaining the sample, (ii) assaying gene expression products of the sample, (iii) inputting the data into a trained algorithm within a computer, and (iv) electronically outputting a report that identifies the sample of tissue as positive or negative for lung cancer. The gene expression products may be assayed by sequencing, array hybridization, or nucleic acid amplification. The gene expression product assay data corresponds to the presence of absence of one or more gene fusions in the data wherein one or more gene fusions are associated with a risk of developing lung cancer. The trained classifier algorithm generates a classification of the sample of tissue as positive or negative for lung cancer. The sample may be obtained by bronchoscopy. The sample may be obtained by fine needle aspiration. The sample may comprise a mucous epithelial tissue, a nasal epithelial tissue, a lung epithelial tissue, or any combination thereof. The sample may comprise epithelial tissue obtained along an airway of the subject.

The trained classifier algorithm may be trained by a training set. The trained classifier algorithm may be trained with a training set that is independent of the sample. The training set may comprise samples benign for a lung condition and samples malignant for the same lung condition. The training data set may comprise samples obtained from subjects associated with a risk of developing lung cancer. The trained classifier algorithm may comprise a first filter and a second filter wherein the first filter identifies gene fusion candidates and the second filter identifies refined gene fusion candidates to generate the classification. The first filter may filter data by the number of junction reads. The number of junction required may be greater than or equal to three. The second filter may identify gene fusion candidates by scoring (i) a prevalence value of a gene fusion in both bronchial brushing lung cancer and TBB benign patient cohorts; (ii) a risk ratio (RR); and (iii) a positive predicative value (PPV). The prevalence value may comprise a detection number and a detection percent. The detection number may be the number of samples in which the gene fusion was detected. The detection percent may be a percent of frequency of gene fusion detection in non-lung cancer samples. The risk ratio may be calculated from the formula (RR)=(M_{f}/N_{f})/(M_{nof}/N_{nof}) wherein wherein M_{f} is the number of malignant cancer samples in which the gene fusion is detected, M_{nof} is the number of malignant cancer samples in which the gene fusion is not detected, B_{f} is the number of benign cancer samples in which the gene fusion is detected, B_{nof} is the number of malignant cancer samples in which the gene fusion not detected, N_{f} is the sum of M_{f} and B_{f}, and N_{nof} is the sum of M_{nof} and R_{nof}. The positive predictive value (PPV) may be calculated from the formula (PPV)=(N(M_{f})/N(N_{f})) wherein N(M_{f}) is a function of M_{f} and N(N_{f}) is a function of N_{f}. The second filter may require a PPV>0.5. The second filter may require a RR>1.

A gene fusion may be considered to be associated with lung cancer if the RR is greater than 1 among all samples and if a gene fusion was detected at least once in the primary analysis with a risk ratio greater than one. A gene fusion may be considered to be associated with lung cancer if the PPV is greater than 0.5 among all samples. Gene fusions may include one or more members selected the group consisting of ASCC2_DEK, EVPL_HLA-DRB5, BPIFB1_FAM73B, ARID4B_TMX4, TFDP2_XRN1, H6PD_SPSB1, APP_GTF3C1, PPL_SAA1, LRP10_MUC5AC, C1orf87_CD36, HLA-DRB5_ZNF497, IKZF5_MUC5AC, EPPK1_LHCGR, C19orf33_MRPL30, EHD3_KIAA1429, MYO9B_WASF2, GGNBP2_MYO19, CCDC64B_CCDC78, POLR1A_REEP1, ATXN3_MAML2, CPSF6_FAM203B, MUC16_MUC4, OS9_RYR1, and PPFIA3_TRPM4.

Another aspect of the present disclosure provides a method for processing or analyzing a sample of epithelial tissue from a respiratory tract of a subject, comprising (a) providing said sample of epithelial tissue from a respiratory tract of the subject, wherein the sample comprises gene expression products; (b) assaying the gene expression products of the sample by sequencing, array hybridization, sequence hybridization, or nucleic acid amplification to yield data; (c) in a programmed computer, applying a score factor to said data to yield a classification of the sample as positive or negative for lung cancer; and (d) electronically outputting a report that identifies the classification of the sample. The score factor can be applied to an expression value of genes associated with exposure to inhaled medications.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 outlines a method of classifying an epithelial tissue sample, from a subject's respiratory tract, as positive or negative for lung cancer based on sequencing data derived from the sample.

FIG. 2 illustrates an overview of the bronchial brushing samples analyzed.

FIG. 3 shows a diagram of a workflow to detect lung cancer associated fusions.

FIG. 4 illustrates a contingency table to define risk ratio and positive predicative value of a fusion f.

FIG. 5 illustrates a table of 26 fusions identified by a method disclosed herein to be associated with lung cancer.

FIG. 6 shows expression levels of a LRP10 fusion.

FIG. 7 shows expression levels of a MUC5AC fusion

FIG. 8 shows expression levels of a C1orf87 fusion.

FIG. 9 shows expression levels of a CD36 fusion.

FIG. 10 shows a computer system that is programmed or otherwise configured to implement methods provided herein.

FIGS. 11A-11B show the calculated specificity of and sensitivity of the predicted classification of each sample, with the calculated specificity and sensitivity of the predicted classification of association with inhaled medications highlighted within boxes. The x-axis shows the classifier's calculated value by classifier type: byPvalue, hcProp0.5, or hpProp0.5. The y-axis shows the specificity value or the sensitivity value.

FIG. 12 shows the score given to each sample by the classifier (Clin7v2.hpProp0.5), and whether that sample came from a benign sample or a malignant sample.

FIG. 13 shows a comparison of the calculated specificity of the predicted classification of each sample by the classifier without the inhaled medication covariate (Clin7.v2) and with the inhaled medication covariate (Clin8.v2).

FIG. 14A-14B show a comparison of calculated specificity and sensitivity of the predicted classification of each sample by the classifier without the inhaled medication covariate (Clin7.v2) and with the inhaled medication covariate (Clin8.v2). The difference between calculated specificity and sensitivity of the predicted classification of association with inhaled medication by each classifier is highlighted with an arrow pointing out the difference between the values corresponding to samples classified as associated with inhaled medication (light grey arrow) and not associated with inhaled medication (dark grey arrow).

FIG. 15 shows a comparison of the calculated specificity of the predicted classification of each sample by the classifier without the inhaled medication covariate after processing sample data with a score shift (WithinCV score shift) and without a score shift (Raw score).

FIG. 16A-16B show a comparison of calculated specificity and sensitivity of the predicted classification of each sample by the classifier without the inhaled medication covariate with a score shift and without a score shift. The difference between the calculated specificity and sensitivity of the predicted classification of association with inhaled medication with and without the score shift is highlighted with an arrow pointing out of the difference between the values corresponding to samples classified as associated with inhaled medication (light grey arrow) and not associated with inhaled medication (dark grey arrow).

FIG. 17A-17D show the calculated specificity, sensitivity, area-under-the-curve (AUC), and score given by the genomic sequencing classifier (GSC) to benign and malignant samples based on whether the sample is or is not associated with inhaled medications.

FIG. 18A-18D show the calculated specificity, sensitivity, area-under-the-curve (AUC), and score given by the genomic sequencing classifier (GSC) to benign and malignant samples based on whether the sample is or is not associated with COPD.

FIG. 19A-19D show the calculated specificity, sensitivity, area-under-the-curve (AUC), and score given by the genomic sequencing classifier (GSC) to benign and malignant samples based on whether the sample is or is not associated with inhaled medications and COPD.

FIG. 20 shows a table naming which registry the samples came from that were used in the inhaled medication study, whether they were associated with an inhaled medication, the calculated sensitivity of the classifier with the data, the calculated specificity of the classifier with the data, the negative predictive value (NPV) of the classifier, the positive predictive value (PPV) of the classifier, the Risk of Malignancy (ROM) and the number of True Negatives (TN), True Positives (TP), False Positives (FP) and False Negatives (FN).

FIG. 21 shows a graph visualization of the expression pattern of samples from the Airway Epithelium Gene Expression in the Diagnosis of Lung Cancer (AEGIS) registry, the Detection of Early Lung Cancer Among Military Personnel (DECAMP) registry, and the Registry. The oval highlights samples from AEGIS and DECAMP that have overlapping expression profiles. The circle highlights samples from the Registry that with expression profiles that do not overlap with either AEGIS or DECAMP.

FIG. 22A-22B show the expression level of genes present in basal cells grouped by the registry source of the samples from which the gene expression data was obtained, using a Unified Assay (UA). FIG. 22B shows the expression levels of genes present in basal cells grouped by the registry source of the samples from which the gene expression data was obtained, using a microarray.

FIG. 23A-23B show the expression levels of HBB (beta-globulin) as an indication of blood contamination from samples grouped by the registry source of the samples from which the gene expression was obtained, using a Unified Assay (UA), or a Microarray.

FIG. 24 shows the expression levels of HBB as an indication of blood contamination from samples grouped by whether, after classification by the classifier and further validation, the sample was a True Negative (TN), False Positive (FP), True Positive (TP), False Negative (FN), or nondiagnostic.

FIG. 25 shows a graph visualization of the correlation between the gene expression data from each sample in the Registry of genes present in basal cells versus HBB. The correlation coefficient was calculated to be 0.35 between the sets of expression data.

FIG. 26A-26C show graph visualizations of the expression pattern of samples from the AEGIS registry, the DECAMP registry, and the Registry. FIG. 26A shows a graph visualization of the expression pattern of samples from all three registries. FIG. 26B shows a graph visualization of the expression pattern of samples from the Registry only. FIG. 26C shows a graph visualization of samples from all three registries, noting the timing of the sample by differentiating those that were collected after a clinical sample was taken and those taken before a clinical sample was taken.

FIG. 27A-27B show the proportion of samples from the Registry that were taken before or after a clinical sample. FIG. 27C shows the expression level of HBB in samples from the Registry, noting whether they were taken after or before a clinical sample and if they were associated with a sample that was benign, nondiagnostic, or malignant, as well as the cancer subtype.

FIG. 28 shows a table naming which registry the samples came from that were used in the collection timing study, whether they were associated with an inhaled medication, the calculated sensitivity of the classifier with the data, the calculated specificity of the classifier with the data, the negative predictive value (NPV) of the classifier, the positive predictive value (PPV) of the classifier, the Risk of Malignancy (ROM) and the number of True Negatives (TN), True Positives (TP), False Positives (FP) and False Negatives (FN).

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “gene fusion” as used herein, generally refers to a hybrid gene formed by two previously separate genes. A gene fusion may be a chimeric molecule derived from two separate genes. The two separate genes may be considered a donor gene and an acceptor gene. A donor gene is generally located upstream of an acceptor gene. A gene fusion may arise from a splicing event such as a chromosomal aberration. A chromosomal aberration may be, for example, an inversion, deletion or translocation within a chromosome or between chromosomes. The expression product of a gene fusion may result in an expression product with a different function as compared to the expression products of the donor gene and acceptor gene. Alternatively, a gene may be fused to a strong promoter, resulting in a proto-oncogene of which the expression product is an oncogene.

The term “respiratory tract,” as used herein, generally refers to tissue found along the nose, mouth, throat, trachea, airway, bronchi, and/or lungs of a subject.

The term “homology,” as used herein, generally refers to calculations of homology or percent homology between two or more nucleotide or amino acid sequences that may be determined by aligning the sequences for comparison purposes (e.g., gaps can be introduced in the sequence of a first sequence). Nucleotides at corresponding positions may then be compared, and the percent identity between the two sequences may be a function of the number of identical positions shared by the sequences (i.e., % homology=# of identical positions/total # of positions×100). For example, if a position in the first sequence is occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. The length of a sequence aligned for comparison purposes may be at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 95%, of the length of the reference sequence.

The term “lung cancer,” as used herein, generally refers to a cancer or tumor of a lung or lung-associated tissue. For example, lung cancer may comprise a non-small cell lung cancer, a small cell lung cancer, a lung carcinoid tumor, or any combination thereof. A non-small cell lung cancer may comprise an adenocarcinoma, a squamous cell carcinoma, a large cell carcinoma, or any combination thereof. A lung carcinoid tumor may comprise a bronchial carcinoid. A lung cancer may comprise a cancer of a lung tissue such as a bronchiole, an epithelial cell, a smooth muscle cell, an alveoli, or any combination thereof. A lung cancer may comprise a cancer of a trachea, a bronchius, a bronchiole, a terminal bronchiole, or any combination thereof. A lung cancer may comprise a cancer of a basal cell, a goblet cell, a ciliated cell, a neuroendocrine cell, a fibroblast cell, a macrophage cell, a Clara cell, or any combination thereof.

The term “fragment,” as used herein, generally refers to a portion of a sequence, such as a subset that may be shorter than a full length sequence. A fragment may be a portion of a gene.

The term “amplification”, as used herein, generally refers to any process of producing at least one copy of a nucleic acid molecule. The terms “amplicons” and “amplified nucleic acid molecule” refer to a copy of a nucleic acid molecule and can be used interchangeably.

The term “machine learning algorithm” as used herein, generally refers to a computationally-based methodology, including an algorithm(s) and/or statistical model(s), that may perform a specific task without using explicit instructions, such as, for example, relying on patterns and inference. A machine learning algorithm may be an algorithm that has been trained or may be trained on at least one training set, which may be used to characterize a biomolecule profile. A machine learning algorithm may be a classifier of a disease or tissue type. A biomolecule profile may be a gene expression profile (e.g., a profile or mRNA or cDNA molecules derived from mRNA). A biomolecule profile may be a nucleic acid sequence profile, e.g., a profile of amino acid sequences, a profile of RNA and DNA sequences, a profile of DNA sequences, a profile of RNA sequences, or any combination thereof. The signals corresponding to certain expression levels, which may be obtained by, e.g., microarray-based hybridization or sequencing assays, may be t subjected to the classifier algorithm to classify the expression profile. Machine learning may be supervised or unsupervised. Supervised learning generally involves “training” a classifier to recognize the distinctions among classes and then “testing” the accuracy of the classifier on an independent test set. For new, unknown samples the classifier can be used to predict the class in which the samples belong.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Overview

Gene fusions may be hybrid genes formed by two previously separate genes. Gene fusions may be strong driver mutations for cancer and may play important roles in tumorigenesis. Gene fusions are typically identified in biological samples extracted from a diseased location. However, for lung cancer, extracting samples from the nodules may be challenging or lead to undesirable consequences.

Disclosed here are systems and methods for detecting gene (or genetic) fusions to determine a risk of disease or identify the disease. Such disease may be a lung disease, such as lung cancer. Also disclosed herein are systems and methods for determine a risk of the disease by assaying one or more samples from a subject to generate data, and processing the data to determine the risk of the disease based at least in part on the presence or absence of one or more gene fusion(s).

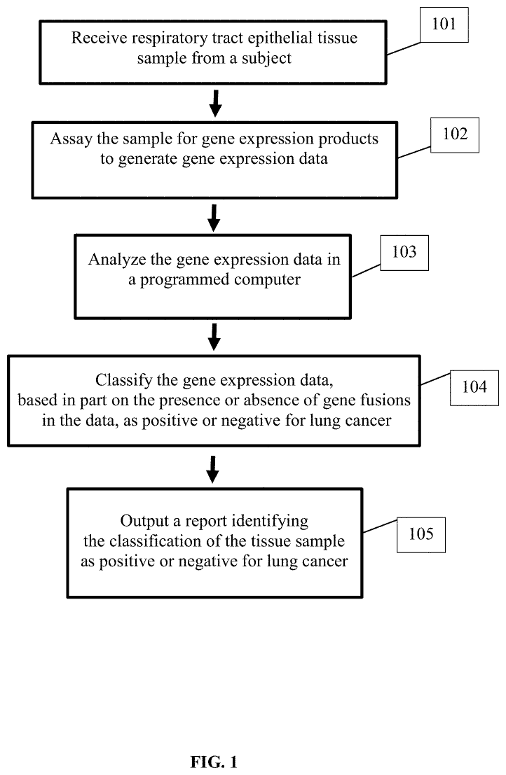

FIG. 1 outlines a method of classifying an epithelial tissue sample as positive or negative for lung cancer based on sequencing data derived from the epithelial tissue sample. In operation 101, a sample may be first received from a subject. Next, in operation 102, the sample may be assayed to generate nucleic acid sequence data, e.g., gene expression data. Next, in operation 103, the gene expression data may be analyzed by a programmed computer. The programmed computer may then classify the gene expression data, based in part on the presence of absence of gene fusion data, as positive or negative for lung cancer, as shown in operation 104. In operation 105, a report may be output indicating the classification for lung cancer.

A sample may be provided or obtained from a subject. The sample may comprise cells obtained from a portion of an airway, such as epithelial cells obtained from a portion of an airway. The sample may be a tissue sample removed from the subject, such as a tissue brushing, a swabbing, a tissue biopsy, an excised tissue, a fine needle aspirate, a tissue washing, a cytology specimen, a bronchoscopy, or any combination thereof. The sample may be provided or obtained from a subject who is using one or more inhaled medications. The inhaled medications may include, for example, bronchodilators, steroids, or a combination thereof. The sample may be obtained or provided after a clinical sample is extracted from the subject. The clinical sample may be a sample that is obtained by needle aspiration or biopsy.

The sample may comprise cells obtained from a respiratory tract of the subject. The sample may be a nasal tissue, a bronchial tissue, a lung tissue, an esophageal tissue, a larynx tissue, an oral tissue or any combination thereof. The sample may comprise cells obtained from a nasal tissue, a bronchial tissue, a lung tissue, an esophageal tissue, a larynx tissue, an oral tissue or any combination thereof. The sample may be suspected or confirmed of evidencing a disease or disorder, such as a cancer or a tumor. For instance, an airway brushing sample (e.g., a bronchial brushing sample) may be obtained from a subject after results from a bronchoscopy are found to be inconclusive. In collecting an airway brushing sample, multiple brushing samples may be collected from a given field in the subject's airway.

Samples that are known or confirmed as evidencing a disease or disorder may be used for machine learning algorithm training purposes.

The sample obtained may have a variety of pathologies. The sample may be cytologically indeterminate. The sample may be cytologically normal. The sample may be an ambiguous or suspicious sample, such as a sample obtained by fine needle aspiration, a bronchoscopy, or other small volume sample collection method. The sample may be derived from an intact region of a patient's body receiving cancer therapy, such as radiation. The sample may be a tumor in a patient's body. The sample may comprise cancerous cells, tumor cells, malignant cells, non-cancerous cells (e.g., normal or benign cells), or a combination thereof. The sample may comprise invasive cells, non-invasive cells, or a combination thereof.

The sample may be a nasal tissue, a tracheal tissue, a lung tissue, a pharynx tissue, a larynx tissue, a bronchus tissue, a pleura tissue, an alveoli tissue, or any combination or derivative thereof. The sample may be a plurality of cells (e.g., epithelial cells) obtained by bronchial brushing. The sample may be a plurality of cells (e.g., lung tissue) obtained by biopsy. The sample may be a secretion comprising a plurality of cells (e.g., epithelial cells) obtained by swab or irrigation of a mucus membrane.

Samples may include samples obtained from: a subject having a pre-existing benign lung disease; a subject having chronic pulmonary infections; a subject having a suppressed immune system; a subject having an increased hereditary risk of developing a lung condition; a non-smoker having environmental exposure; or any combination thereof. Samples may be obtained from a plurality of different countries.

The sample may be an isolated and purified sample. The sample may be a freshly isolated sample. Cells from the freshly isolated sample may be isolated and cultured. The sample may comprise one or more cells. An isolated sample may comprise a heterogeneous mixture of cells. A sample may be purified to comprise a homogeneous mixture of cells. The sample may comprise at least about 100 cells, 1,000 cells, 5,000 cells, 10,000 cells, 20,000 cells, 30,000 cells, 40,000 cells, 50,000 cells, 60,000 cells, 70,000 cells, 80,000 cells, 90,000 cells, 100,000 cells, 150,000 cells, 200,000 cells, 250,000 cells, 300,000 cells, 350,000 cells, 400,000 cells, 450,000 cells, 500,000 cells, 550,000 cells, 600,000 cells, 650,000 cells, 700,000 cells, 750,000 cells, 800,000 cells, 850,000 cells, 900,000 cells, 950,000 cells, or more. The sample may comprise from about 30,000 cells to about 1,000,000 cells. The sample may comprise from about 20,000 cells to about 50,000 cells. The sample may comprise from about 100,000 cells to about 400,000 cells. The sample may comprise from about 400,000 cells to about 800,000 cells.

The sample may be collected from the same subject more than one time. Periodic sample collection may be performed to monitor a subject that is identified as being at risk for lung cancer or lung disease. For example, a first sample may be collected from a subject and a second sample may be collected about 1 year after the first sample has been collected. Samples may be collected from the same subject about: bi-weekly, weekly, bi-monthly, monthly, bi-yearly, yearly, every two years, every three years, every four years, or every five years. Samples may be collected annually from a subject. Results from the second sample may be compared to results of a first sample to monitoring a disease progression in the subject, an efficacy of a prescribed treatment or therapy, or a change in a risk of developing a condition, or any combination thereof.

The sample may be then assayed by methods described herein to generate nucleic acid sequence data. The nucleic acid sequence data may correspond to one or more gene fusions. Such gene fusions may be strong driver mutations for cancer and play important roles in tumorigenesis. As non-limiting examples, gene fusions may include, but are not limited to, at least 1, 2, 3, 4, 5, 6, 7, 8, 9, 10, or more gene fusions selected from the group consisting of ASCC2_DEK, EVPL_HLA-DRB5, BPIFB1_FAM73B, ARID4B_TMX4, TFDP2_XRN1, H6PD_SPSB1, APP_GTF3C1, PPL_SAA1, LRP10_MUC5AC, C1orf87_CD36, HLA-DRB5_ZNF497, IKZF5_MUC5AC, EPPK1_LHCGR, C19orf33_MRPL30, EHD3_KIAA1429, MYO9B_WASF2, GGNBP2_MYO19, CCDC64B_CCDC78, POLR1A_REEP1, ATXN3_MAML2, CPSF6_FAM203B, MUC16_MUC4, OS9_RYR1, and PPFIA3_TRPM4.

Gene fusions may be detected by assaying, such as by sequencing, sequencing identification, or sequence hybridization. Sequencing be massively parallel array sequencing (e.g., Illumina), single molecule sequencing (Pacific Biosciences of California or Oxford Nanopore), whole genome sequencing, or targeted sequencing. Sequence identification may be performed using microarray hybridization, fluorescent in situ hybridization (FISH), or polymerase chain reaction (PCR). Sequencing or sequence identification may involve sample preparation, such as reverse transcription and/or enrichment. Sequence hybridization may be performed by a nCounter Dx Analysis system. Sequence hybridization may comprise the use of a barcode to identify target genes. Each target may be identified by the hybridization of a capture probe and a reporter probe to the target molecule. The capture probe may comprise a capture molecule (e.g., biotin) and a sequence complementary to the target sequence. The reporter probe may comprise a barcode or reporter molecule and a sequence complementary to the target sequence. The target sequence may be mRNA. The capture probe can be used to isolate the hybridized target sequence. A digital analyzer may be used to identify and count the barcode sequence attached to the reporter probe.

Nucleic acid sequences, such as gene fusions, among other target sequences, may be enriched in a nucleic acid sample. Nucleic acid sequences may be enriched prior to detection, e.g., to enhance the detection of fusions present at a low abundance relative to other sequences in a nucleic acid sample.

The method may include first contacting at least one target sequence within a sample comprising gene expression products with one or more nucleic acid probes under conditions where the one or more nucleic acid probes hybridizes to at least one target sequence. The hybridized probe-target complex may be amplified (e.g., using polymerase chain reaction (“PCR”)) to enrich a target sequence relative to other nucleic acid molecule sequences in a sample. The hybridized probe-target complex may be “pulled-down” or isolated from a sample, such that non-hybridized nucleic acid molecules are removed. As a non-limiting example, biotinylated RNA capture probes may hybridize to sequences in a solution targeted for enrichment. Complexes of biotinylated capture probes and complementary target sequences may be isolated from a solution by incubating with streptavidin-coated magnetic beads. The method may include techniques to reduce amplification bias. Techniques to reduce amplification bias may include degenerate primers or targeting amplicons with conserved priming sites.

Methods and systems disclosed herein may use one or more capture probes, a plurality of capture probes, or one or more capture probe sets. The capture probe comprises a nucleic acid binding site (e.g., a sequence complementary to the capture probe). The capture probe may further comprise one or more linkers. The capture probes may further comprise one or more labels. The one or more linkers may attach the one or more labels to the nucleic acid binding site.

A plurality of capture probes or capture probe sets may be used to enrich certain sets or subsets of nucleic acids (e.g., gene fusions) from a sample. Nucleic acids may be enriched using 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more one or more capture probes or capture probe sets. The one or more capture probes or capture probe sets may be different, similar, identical, or a combination thereof.

The one or more capture probes may comprise a nucleic acid binding site that hybridizes to at least a portion of the one or more nucleic acid molecules or variant or derivative thereof in the sample or subset of nucleic acid molecules. The capture probes may comprise a nucleic acid binding site that hybridizes to one or more target sequences (e.g., genes or portions thereof, or gene fusions). The capture probes may hybridize to different, similar, and/or identical genomic regions. The one or more capture probes may be at least about 50%, 55%, 60%, 65%, 70%, 75%, 80%, 85%, 90%, 95%, 97%, 99% or more complementary to the one or more nucleic acid molecules or variant (e.g., fusion, SNP, indel, and the like) or derivative thereof.

Capture probes may comprise one or more nucleotides. The capture probes may comprise 1 or more, 2 or more, 3 or more, 4 or more, 5 or more, 6 or more, 7 or more, 8 or more, 9 or more, 10 or more, 20 or more, 30 or more, 40 or more, 50 or more, 60 or more, 70 or more, 80 or more, 90 or more, 100 or more, 125 or more, 150 or more, 175 or more, 200 or more, 250 or more, 300 or more, 350 or more, 400 or more, 500 or more, 600 or more, 700 or more, 800 or more, 900 or more, or 1000 or more nucleotides. The capture probes may comprise about 100 nucleotides. The capture probes may comprise between about 10 to about 500 nucleotides, between about 20 to about 450 nucleotides, between about 30 to about 400 nucleotides, between about 40 to about 350 nucleotides, between about 50 to about 300 nucleotides, between about 60 to about 250 nucleotides, between about 70 to about 200 nucleotides, or between about 80 to about 150 nucleotides. In some aspects of the disclosure, the capture probes comprise between about 80 nucleotides to about 100 nucleotides.

The plurality of capture probes or the capture probe sets may comprise two or more capture probes with identical, similar, and/or different nucleic acid binding site sequences, linkers, and/or labels. For example, two or more capture probes comprise identical nucleic acid binding sites. In another example, two or more capture probes comprise similar nucleic acid binding sites. In yet another example, two or more capture probes comprise different nucleic acid binding sites. The two or more capture probes may further comprise one or more linkers. The two or more capture probes may further comprise different linkers. The two or more capture probes may further comprise similar linkers. The two or more capture probes may further comprise identical linkers. The two or more capture probes may further comprise one or more labels. The two or more capture probes may further comprise different labels. The two or more capture probes may further comprise similar labels. The two or more capture probes may further comprise identical labels.

A probe may comprise a target specific sequence and an adapter sequence. The adapter sequence may also comprise a sample identifier. The method may include detecting the presence of the adapter sequence in the modified probe, thereby identifying the target sequences in the sample comprising gene expression products. Detecting the presence of the adapter sequence may be by the amplification of the sequence. The gene expression products may be RNA or DNA. The adapter sequence may be configured to attach to a solid support, such as a bead, via the adapter sequence.

Nucleic acid molecules may be amplified. The amplification reactions may comprise PCR-based methods, non-PCR based methods, or a combination thereof. Examples of non-PCR based methods may include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. PCR-based methods may include, but are not limited to, PCR, HD-PCR, Next Gen PCR, digital RTA, or any combination thereof. Additional PCR methods may include, but are not limited to, linear amplification, allele-specific PCR, Alu PCR, assembly PCR, asymmetric PCR, droplet PCR, emulsion PCR, helicase dependent amplification HDA, hot start PCR, inverse PCR, linear-after-the-exponential (LATE)-PCR, long PCR, multiplex PCR, nested PCR, hemi-nested PCR, quantitative PCR, RT-PCR, real time PCR, single cell PCR, and touchdown PCR.

RNA sequencing (such as exome enriched RNA sequencing or the sequencing of cDNA obtained from RNA) typically generates short sequence fragments. RNA can be sequenced by first undergoing reverse transcription into cDNA (i.e. RT-qPCR, RT-PCR, qPCR). Following reverse transcription, the cDNA can be sequenced. Each fragment, or “read”, of a cDNA molecule can be used to measure levels of gene expression.

Sequence identification methods may include sequence hybridization methods such as NanoString. Sequencing methods may include, but are not limited to: high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Nova Seq (Illumina), Digital Gene Expression (Helicos), Single Molecule Sequencing by Synthesis (SMSS)(Helicos), massively-parallel sequencing, Clonal Single Molecule Array (Solexa), shotgun sequencing, Maxim-Gilbert sequencing, primer walking, sequencing using PacBio, SOLiD, Ion Torrent, or Nanopore platforms and any other sequencing methods.

Sequencing may include sequencing technologies having increased throughput as compared to traditional Sanger- and capillary electrophoresis-based approaches, for example with the ability to generate hundreds of thousands of relatively small sequence reads at a time. Some examples of sequencing techniques include, but are not limited to, sequencing by synthesis, sequencing by ligation, and sequencing by hybridization.

Additional techniques may be used to detect various biomarkers in addition to gene fusions (e.g., DNA, cDNA, transcripts thereof, and related peptide sequences).

Epigenetic biomarkers (such as DNA methylation, such as 5-hydroxymethylated cytosine, 5-methylated cytosine, 5-carboxymethylated cytosine, or 5-formylated cytosine) may be detected by sequencing, microarrays, PCR, RT-PCR, qPCR, mass spectrometry (MS), Chromatin Immunoprecipitation (ChIP) or any combination thereof.

Transcriptomic biomarkers (such as RNA expression levels) may be detected by sequencing, microarrays, PCR, or any combination thereof.

Proteomic biomarkers (such as a presence of a protein) may be detected by protein arrays, immunohistochemical staining (IHC), enzyme-linked immunoabsorbance assays (ELISA), mass spectrometry, immunohistochemistry, blotting or a combination thereof.

A fusion sequence may contain a fragment of a reference sequence, or other pre-determined sequence, such as a specific junction, specific donor, or specific acceptor sequences. A fusion sequence may contain one or more junction reads.

A junction read may be a read correlating to (e.g., related to or associated with) an exon-exon junction. A junction may refer to a region where a splicing event takes place. For example, a gene donating an exon to a fusion may be present at the 5′ end of an intron and an exon-intron junction where the splice event occurs may be present at the 3′ end of an intron. An exon-exon junction may be the product of gene splicing in which a chimeric gene fusion is formed from the splicing of two genes, the donor gene and the recipient gene. To identify a gene fusion as such, at least three junction reads may be found for each gene fusion. Junction reads can be aligned with reference sequences to determine the composition of the fusion protein based off homology with reference sequences. A sequence homology may be from about 70% to 100%, from about 80% to 100%, from about 90% to 100%, or be from about 95% to 100%. The sequence homology may be from about 70% to 99%. In some cases, a sequence homology may be from about 80% to 99%. In some cases, a sequence homology may be from about 90% to 99%. In some cases, a sequence homology may be from about 95% to 99%.

Sequence homology may be determined in various ways. The two sequences can be genes, nucleotides sequences, protein sequences, peptide sequences, amino acid sequences, or fragments thereof. A BLAST® search may determine homology between the two sequences. Comparison of the two sequences can be accomplished by various methods such as, for example, using a mathematical algorithm. A non-limiting example of such a mathematical algorithm is described in Karlin, S. and Altschul, S., Proc. Natl. Acad. Sci. USA, 90-5873-5877 (1993). An algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another example, the percent identity between two amino acid sequences can be accomplished using, for example, the GAP program in the GCG software package (Accelrys, Cambridge, UK).

A classifier algorithm may be used to garner insight into whether a biological sample evidences a presence, absence, or suspicion of cancer cells. The classifier algorithm may be used to analyze biomolecule information (e.g., DNA sequences, RNA sequences, and/or expression profiles) in samples that are otherwise inconclusive for cancer to determine whether the subject from which the sample was obtained has a pre-test high risk or pre-test low risk for cancer. As a non-limiting example, a bronchoscopy taken from a subject's lung nodule (initially detected via computerized tomography (CT) scan) may be determined to be inconclusive. Such a patient may be at a pre-test “intermediate” risk for lung cancer. Additional bronchial brushing samples may be taken from the subject and the nucleic acid molecules in these samples may be analyzed by sequencing to yield sequence information detect one or more gene fusions. The classifier may be used to process the sequence information and down-classify the subject's sample (which may initially be inconclusive or intermediate risk) as post-test “low risk” for lung cancer or up-classify the subject as post-test “high-risk” for lung cancer.

For example, a pre-test risk of malignancy is low if it is less than about 10%, 9%, 8%, 7%, 6%, 5%, 4%, 3%, 2%, or 1%. A pre-test risk of malignancy is intermediate if it is greater than about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, or 59%, and less than about 60%. A pre-test risk of malignancy is intermediate it is less about 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, or 11%, and greater than about 10%. A pre-test risk of malignancy is high if it is greater than about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

For example, a post-test risk of malignancy is low if it is less than about 10%, 9%, 8%, 7%, 6%, 5%, 4%, 3%, 2%, or 1%. A post-test risk of malignancy is intermediate if it is greater than about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, or 59%, and less than about 60%. A post-test risk of malignancy is intermediate it is less about 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, or 11%, and greater than about 10%. A post-test risk of malignancy is high if it is greater than about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

For example, post-test risk of malignancy is very low if it is less than about 1%, 0.9%, 0.8%, 0.7%, 0.6%, 0.5%, 0.4%, 0.3%, 0.2%, or 0.1%. A post-test risk of malignancy is low if less than about 10%, 9%, 8%, 7%, 6%, 5%, 4%, 3%, 2%, or 1.5%, and great than about 1%. A post-test risk of malignancy is intermediate if it is greater than about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, or 59%, and less than about 60%. A post-test risk of malignancy is intermediate it is less about 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, or 11%, and greater than about 10%. A post-test risk of malignancy is high if it is greater than about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, or 89%, and less than about 90%. A post-test risk of malignancy is very high if it is greater than about 90%, 91%, 92%, 93%, 94%, 95%, 96%, 97%, 98%, or 99%.

A classifier algorithm may be trained with one or more training samples. The training samples may include covariates such as whether the sample was taken from an subject using inhaled medications, including for example bronchodilators, steroids, or a combination of bronchodilators and steroids, whether the sample was taken before or after a clinical sample, the smoking history of the subject, the gender of the subject, the current smoking status of the subject, etc. The classifier algorithm may be trained with a set of training samples that are independent of the sample analyzed by the classifier algorithm. The classifier algorithm may be trained with one or more different types of training samples. The classifier algorithm may be trained with at least two different types of training samples, such as a bronchial brushing sample and a fine needle aspiration. In another example, the training set may comprise samples benign for a lung condition and samples malignant for a lung condition. The training set may comprise samples that are determined to be benign for a lung condition and samples that are malignant for at least that same lung condition. A training data set may comprise samples obtained from subjects associated with a risk of developing lung cancer, examples include but are not limited to subjects with a history of smoking cigarettes or having an exposure to asbestos.

Training samples may be samples that are obtained from a subject prior to or following collection of a clinical sample (e.g., a biopsy or needle aspirate), or both. The training samples obtained before, after, or both before and after obtaining a clinical sample may be a bronchial brushing sample, a buccal sample, or a bronchoscopy sample.

Training samples may include sample(s) that are from a subject(s) taking one or more inhaled medications.

A classifier algorithm may be trained with at least three different types of training samples, such as a surgical biopsy, fine needle aspiration, buccal samples, and bronchial brushing. The classifier algorithm may be trained with at least three different types of training samples, such as a surgical biopsy, fine needle aspiration, and an image obtained from a CT scan. The classifier algorithm may be trained with at least four different types of training samples, such as a surgical biopsy, fine needle aspiration, a bronchial brushing, and an image obtained from a CT scan. The classifier algorithm may be trained with bronchial brushing samples, buccal samples, and bronchoscopy samples labeled as normal, benign, cancerous, malignant, or any combination thereof.

The methods and systems disclosed herein may classify a sample obtained from a subject as positive or negative for a lung condition (e.g., lung cancer) with high sensitivity, specificity, and/or accuracy. The sample may be classified as positive or negative for a lung condition (e.g., lung cancer) with a specificity of at least about 60% 70%, 80%, 85%, 90%, 95%, 99%, or greater. The sample may be classified as positive or negative for a lung condition (e.g., lung cancer) with a sensitivity of at least about 60% 70%, 80%, 85%, 90%, 95%, 99%, or greater. The sample may be classified as positive or negative for a lung condition (e.g., lung cancer) with an accuracy of at least about 60% 70%, 80%, 85%, 90%, 95%, 99%, or greater.

Training samples used to train and validate a trained classifier algorithm may be greater than or equal to about: 100 samples, 200 samples, 300 samples, 400 samples, 500 samples, 600 samples, 700 samples, 800 samples, 900 samples, 1000 samples, 1100 samples, 1200 samples, 1300 samples, 1400 samples, 1500 samples, 1600 samples, 1700 samples, 1800 samples, 1900 samples, 2000 samples, or more (for example 1950 samples obtained from different subjects). In some cases, training samples may comprise from about 100 samples to about 200 samples. In some cases, training samples may comprise from about 100 samples to about 300 samples. In some cases, training samples may comprise from about 100 samples to about 400 samples. In some cases, training samples may comprise from about 100 samples to about 500 samples. In some cases, training samples may comprise from about 100 samples to about 600 samples. In some cases, training samples may comprise from about 100 samples to about 700 samples. In some cases, training samples may comprise from about 100 samples to about 800 samples. In some cases, training samples may comprise from about 100 samples to about 900 samples. In some cases, training samples may comprise from about 100 samples to about 1000 samples. In some cases, training samples may comprise from about 100 samples to about 1500 samples. In some cases, training samples may comprise from about 100 samples to about 2000 samples. In some cases, training samples may comprise from about 100 samples to about 3000 samples. In some cases, training samples may comprise from about 100 samples to about 4000 samples. In some cases, training samples may comprise from about 100 samples to about 5000 samples.

Training samples may be independent of the sample analyzed by the classifier algorithm. Training samples may be obtained from one or more subjects. Subject may include subjects having a different country of birth. Subject may include subject having a different place of residence. Training samples may represent at least about: 2, 3, 4, 5, 6, 7, 8, 9, 10, 11, 12, 13, 14, 15, 16, 17, 18, 19, or 20 different countries of birth. Training samples may represent at least about 3 different countries of birth. Training samples may represent at least about 5 different countries of birth. Training samples may represent at least about 10 different countries of birth. Training samples may represent from about 2 to about 10 different countries of birth. Training samples may represent from about 3 to about 15 different countries of birth. Training samples may represent from about 2 to about 20 different countries of birth. Training samples may represent at least about: 2, 3, 4, 5, 6, 7, 8, 9, 10, 11, 12, 13, 14, 15, 16, 17, 18, 19, or 20 different countries of residence. Training samples may represent at least about 3 different countries of residence. Training samples may represent at least about 5 different countries of residence. Training samples may represent at least about 10 different countries of residence. Training samples may represent from about 2 to about 10 different countries of residence. Training samples may represent from about 3 to about 15 different countries of residence. Training samples may represent from about 2 to about 20 different countries of residence.

Samples in the training set may comprise a plurality of conditions (such as diseases or disease subtypes, consumption of inhaled medication, timing of sample collection relative to clinical sample collection). Samples in an independent test (i.e., independent from the sample being assayed) set may comprise a plurality of conditions (such as disease or disease subtypes). Samples in an independent test set may comprise a least one disease or disease subtype that is different from the samples in the training set. Samples in the training set may comprise a least one disease or disease subtype that is different from the samples in the independent test set. Samples in the independent test set may comprise at least two additional diseases or disease subtypes than the samples in the training set.

Training samples may comprise one or more samples obtained from a subject suspected of having lung cancer, a subject having a confirmed diagnosis of lung cancer, a subject having a pre-existing condition such as a benign lung disease, a subject having lung nodules identified on a LDCT, a subject that may be a non-smoker, a subject that may be a non-smoker with environmental exposure to smoking, a current smoker, a previous smoker, a subject having smoked at least about: 1, 10, 20, 100, 200, 300, 400, 500, 600, 700, 800, 900, 1,000, 2,000, 3,000, 4,000, 5,000, 10,000, 11,000, 12,000, 13,000, 14,000, 15,000, 16,000, 17,000, 18,000, 19,000, 20,000, 30,000, 40,000, 50,000, 60,000, 70,000, 80,000, 90,000, 100,000, 200,000, 300,000, 400,000, 500,000 or more cigarettes or cigars or e-cigarettes in their lifetime, a subject having an increased hereditary risk of developing lung cancer, a subject having a suppressed immune system, a subject having chronic pulmonary infections, or any combination thereof.

Subpopulations from cohorts may drive specific classifier development and validation. Classifiers may be developed for specific population, types of exposures, or combinations thereof. For example, classifiers may be developed for environmental pollution in China or for a genetic predisposition to a lung condition. A genomic classifier may be developed to screen for a lung condition, to diagnose a lung condition, to evaluate a treatment for a lung condition, to monitor a subject's condition, or any combination thereof. A genomic classifier may be developed to determine, based on gene expression levels (e.g., of fusions) whether a sample is benign or malignant for a disease condition (e.g., lung cancer) or a subject's risk of developing a disease condition.

The trained classifier algorithm may comprise a first filter and a second filter wherein the first filter identifies gene fusion candidates and the second filter identifies refined gene fusion candidates to generate said classification.

The first filter may filter data by the number of junction reads. The number of junction required may be greater than or equal to three.

The second filter may identify gene fusion candidates by scoring (i) a prevalence value of a gene fusion in both bronchial brushing lung cancer and TBB benign patient cohorts; (ii) a risk ratio (RR); and (iii) a positive predicative value (PPV).

The prevalence value may comprise a detection number and a detection percent. The detection number may be the number of samples in which the gene fusion was detected. The detection percent may be a percent of frequency of gene fusion detection in non-lung cancer samples. Prevalence values of a gene fusion may be found from public databases. A prevalence value may be calculated by the compilation of gathered data.

The risk ratio may be calculated from the formula (RR)=(M_{f}/N_{f})/(M_{nof}/N_{nof}) wherein M_{f} is the number of malignant cancer samples in which the gene fusion is detected, M_{nof} is the number of malignant cancer samples in which the gene fusion is not detected, B_{f} is the number of benign cancer samples in which the gene fusion is detected, B_{nof} is the number of malignant cancer samples in which the gene fusion not detected, N_{f} is the sum of M_{f} and B_{f}, and N_{nof} is the sum of M_{nof} and B_{nof}.

The positive predictive value (PPV) may be calculated from the formula (PPV)=(N(M_{f})/N(N_{f})) wherein N(M_{f}) is a function of M_{f} and N(N_{f}) is a function of N_{f}. The second filter may require a PPV>0.5. The second filter may require a RR>1.

Intensity values or sequence information generated from nucleic acid sequencing for a sample may be analyzed using feature selection techniques including filter techniques which assess the relevance of features by looking at the intrinsic properties of the data, wrapper methods which embed the model hypothesis within a feature subset search, and embedded techniques in which the search for an optimal set of features may be built into a classifier algorithm.

Filter techniques that may be useful in the methods of the present disclosure include (1) parametric methods such as the use of two sample t-tests, ANOVA analyses, Bayesian frameworks, and Gamma distribution models (2) model free methods such as the use of Wilcoxon rank sum tests, between-within class sum of squares tests, rank products methods, random permutation methods, or TNoM which involves setting a threshold point for fold-change differences in expression between two datasets and then detecting the threshold point in each gene that minimizes the number of misclassifications (3) and multivariate methods such as bivariate methods, correlation based feature selection methods (CFS), minimum redundancy maximum relevance methods (MRMR), Markov blanket filter methods, and uncorrelated shrunken centroid methods. Wrapper methods useful in the methods of the present disclosure include sequential search methods, genetic algorithms, and estimation of distribution algorithms. Embedded methods useful in the methods of the present disclosure include random forest algorithms, weight vector of support vector machine algorithms, and weights of logistic regression algorithms. Bioinformatics, 2007 Oct. 1; 23(19):2507-17 provides an overview of the relative merits of the filter techniques provided above for the analysis of intensity data.

Selected features may then be classified using a classifier algorithm. Illustrative algorithms include but may not be limited to methods that reduce the number of variables such as principal component analysis algorithms, partial least squares methods, and independent component analysis algorithms. Illustrative algorithms further include but may not be limited to methods that handle large numbers of variables directly such as statistical methods and methods based on machine learning techniques. Statistical methods include penalized logistic regression, prediction analysis of microarrays (PAM), methods based on shrunken centroids, support vector machine analysis, and regularized linear discriminant analysis. Machine learning techniques may include bagging procedures, boosting procedures, random forest algorithms, and combinations thereof. See, e.g., Cancer Inform, 2008; 6: 77-97, Clin Transl. Sci., 2011; 4(6):466-477, and J. Phys. Conf Ser., 2018; 971, which is entirely incorporated herein by reference, and J. Proteomics Bioinform., 2010; 3(6):183-190, which is entirely incorporated herein by reference.

Systems and methods of the present disclosure may enable 1) gene expression analysis of a sample containing low amounts and/or low quality of nucleic acids; 2) a significant reduction of false positives and false negatives, 3) a determination of the underlying genetic, metabolic, or signaling pathways responsible for the resulting pathology, 4) the ability to assign a statistical probability to the accuracy of a diagnosis, a risk of developing a condition, a monitoring of changes in a condition, an effectiveness of an interventive therapy, or combinations thereof, 5) the ability to resolve ambiguous results, and 6) the ability to distinguish between lung conditions or sub-types of lung conditions based on the presence of a gene fusion. A sample may contain a low amount of nucleic acids. For example, the sample may contain less than 100 picograms (pg) of DNA, less than 90 pg of DNA, less than 80 pg of DNA, less than 70 pg of DNA, less than 60 pg of DNA, less than 50 pg of DNA, less than 40 pg of DNA, less than 30 pg of DNA, less than 20 pg of DNA, less than 10 pg of DNA. A samples may contain more than 100 pg of DNA, more than 90 pg of DNA, more than 80 pg of DNA, more than 70 pg of DNA, more than 60 pg of DNA, more than 50 pg of DNA, more than 40 pg of DNA, more than 30 pg of DNA, more than 20 pg of DNA, more than 10 pg of DNA. A sample may contain less than 60 nanograms (ng) of RNA, less than 50 ng of RNA, less than 40 ng of RNA, less than 30 ng of RNA, less than 20 ng of RNA, less than 10 ng of RNA, less than 5 ng of RNA. A sample may contain more than 60 ng of RNA, 50 ng of RNA, 40 ng of RNA, 30 ng of RNA, 20 ng of RNA, 10 ng of RNA, 5 ng of RNA. The sample may contain nucleic acids that are of low quality (e.g., as determined by RNA integrity number). Low quality nucleic acid molecules comprising RNA may have an RNA integrity number (“RIN”) of less than 5.0, less than 4.5, less than 4.0, less than 3.5, less than 3.0, less than 2.5, less than 2.0, less than 1.5. Low quality nucleic acid molecules comprising RNA may have a RIN of less than 3.0.

The present disclosure provides for upfront methods of determining the cellular make-up of a particular biological sample so that the resulting molecular profiling signatures may be calibrated against the dilution effect due to the presence of other cell and/or tissue types. This upfront method may be an algorithm that uses a combination of cell and/or tissue specific gene expression patterns as an upfront mini-classifier for one or more or each component of the sample. This algorithm may use the gene expression patterns, or molecular fingerprint, to pre-classify the samples according to their composition and then apply a correction/normalization factor. Then, this data may feed in to an additional classification algorithm which may incorporate that information to aid in a further determination that a sample may be benign or malignant.

Raw gene expression level and alternative splicing data may be improved through the application of algorithms designed to normalize and or improve the reliability of the data. Data analysis may require a computer or other device, machine or apparatus for application of the various algorithms described herein due to the large number of individual data points that may be processed.

In some cases, the robust multi-array Average (RMA) method may be used to normalize the raw data. The RMA method begins by computing background-corrected intensities for each matched cell on a number of microarrays. The background corrected values may be restricted to positive values as described by Irizarry et al. Biostatistics 2003 Apr. 4 (2): 249-64, which is entirely incorporated herein by reference. After background correction, the base-2 logarithm of each background corrected matched-cell intensity may be then obtained. The background corrected, log-transformed, matched intensity on each microarray may be then normalized using the quantile normalization method in which for each input array and each probe expression value, the array percentile probe value may be replaced with the average of all array percentile points, this method may be more completely described by Bolstad et al. Bioinformatics 2003, which is entirely incorporated herein by reference. Following quantile normalization, the normalized data may then be fit to a linear model to obtain an expression measure for each probe on each microarray. Tukey's median polish algorithm (Tukey, J. W., Exploratory Data Analysis. 1977), which is entirely incorporated herein by reference, may then be used to determine the log-scale expression level for the normalized probe set data.

Data may further be filtered to remove data that may be considered suspect. In some embodiments, data deriving from microarray probes that have fewer than about: 1, 2, 3, 4, 5, 6, 7 or 8 guanosine+cytosine nucleotides may be considered to be unreliable due to their aberrant hybridization propensity or secondary structure issues. A microarray probe having more than about 4 guanosine+cytosine nucleotides may be considered unreliable. A microarray probe having more than about 6 guanosine+cytosine nucleotides may be considered unreliable. A microarray probe having more than about 8 guanosine+cytosine nucleotides may be considered unreliable. A microarray probe having from about 4 guanosine+cytosine nucleotides to about 8 guanosine+cytosine nucleotides may be considered unreliable. Similarly, data deriving from microarray probes that have more than about: 8, 9, 10, 11, 12, 13, 14, 15, 16, 17, 18, 19, 20, 21, 22, 23, 24, 25 guanosine+cytosine nucleotides may be considered unreliable due to their aberrant hybridization propensity or secondary structure issues. A microarray probe having more than about 10 guanosine+cytosine nucleotides may be unreliable. A microarray probe having more than about 15 guanosine+cytosine nucleotides may be unreliable. A microarray probe having more than about 20 guanosine+cytosine nucleotides may be unreliable. A microarray probe having more than about 25 guanosine+cytosine nucleotides may be unreliable. A microarray probe having from about 8 guanosine+cytosine nucleotides to about 30 guanosine+cytosine nucleotides may be unreliable. A microarray probe having from about 10 guanosine+cytosine nucleotides to about 30 guanosine+cytosine nucleotides may be unreliable. A microarray probe having from about 12 guanosine+cytosine nucleotides to about 30 guanosine+cytosine nucleotides may be unreliable. A microarray probe having from about 15 guanosine+cytosine nucleotides to about 30 guanosine+cytosine nucleotides may be unreliable.

In some cases, unreliable probe sets may be selected for exclusion from data analysis by ranking probe-set reliability against a series of reference datasets. For example, RefSeq or Ensembl (EMBL) may be considered very high quality reference datasets. Data from probe sets matching RefSeq or Ensembl sequences may in some cases be specifically included in microarray analysis experiments due to their expected high reliability. Similarly data from probe-sets matching less reliable reference datasets may be excluded from further analysis, or considered on a case by case basis for inclusion. In some cases, the Ensembl high throughput cDNA and/or mRNA reference datasets may be used to determine the probe-set reliability separately or together. In other cases, probe-set reliability may be ranked. For example, probes and/or probe-sets that match perfectly to all reference datasets may be ranked as most reliable (1). Furthermore, probes and/or probe-sets that match two out of three reference datasets may be ranked as next most reliable (2), probes and/or probe-sets that match one out of three reference datasets may be ranked next (3) and probes and/or probe sets that match no reference datasets may be ranked last (4). Probes and or probe-sets may then be included or excluded from analysis based on their ranking. For example, one may choose to include data from category 1, 2, 3, and 4 probe-sets; category 1, 2, and 3 probe-sets; category 1 and 2 probe-sets; or category 1 probe-sets for further analysis. In another example, probe-sets may be ranked by the number of base pair mismatches to reference dataset entries. It is understood that there may be many methods understood in the art for assessing the reliability of a given probe and/or probe-set for molecular profiling and the methods of the present disclosure encompass any of these methods and combinations thereof.

Methods of data analysis of gene expression levels or of alternative splicing may further include the use of a feature selection classifier algorithm as provided herein. In some embodiments of the present disclosure, feature selection is provided by use of the LIMMA software package (Smyth, G. K. (2005). Limma: linear models for microarray data. In: Bioinformatics and Computational Biology Solutions using R and Bioconductor, R. Gentleman, V. Carey, S. Dudoit, R. Irizarry, W. Huber (eds.), Springer, New York, pages 397-420), which is entirely incorporated herein by reference.

Methods of data analysis of gene expression levels and or of alternative splicing may further include the use of a pre-classifier algorithm. For example, an algorithm may use a cell-specific molecular fingerprint to pre-classify the samples according to their genetic composition, such as the expression of genes found within a cell (e,g., RNA found in a basal cell or RNA found in a blood cell) and then apply a correction/normalization factor. This data/information may then be fed in to a final classification algorithm which may incorporate that information to aid in a final classification, diagnosis or prognosis, or monitoring evaluation.

Methods of data analysis of gene expression levels and or of alternative splicing may further include the use of a classifier algorithm as provided herein. In some embodiments of the present disclosure a support vector machine (SVM) algorithm, a random forest algorithm, or a combination thereof is provided for classification of microarray data. In some embodiments, identified markers that distinguish samples (e.g., benign vs. malignant, normal vs. malignant, low risk vs. high risk) or distinguish types (e.g., ILD vs. lung cancer) may selected based on statistical significance. In some cases, the statistical significance selection is performed after applying a Benjamini Hochberg correction for false discovery rate (FDR).

### Computer Systems

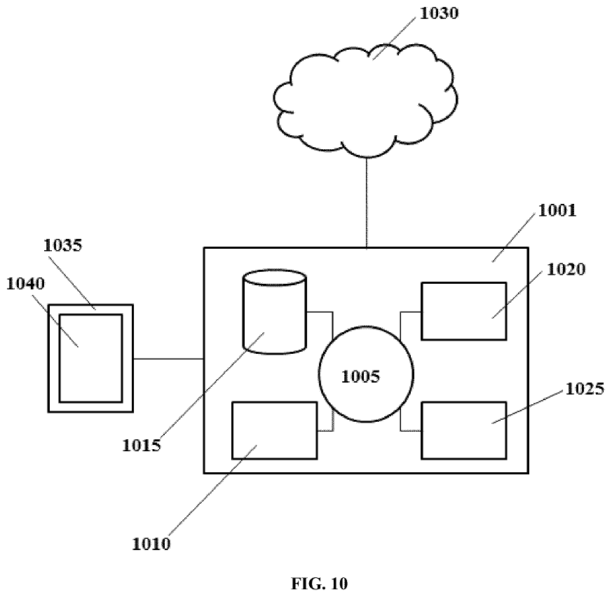

The present disclosure provides computer systems for implementing methods provided herein. FIG. 10 shows an example of a computer system 1001. The computer system 1001 includes a central processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 05 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user (e.g., remote cloud server). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, an electronic output of identified gene fusions. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1005.

### Treatments

Treatment may be provided or administered to a subject based on a classification of subject's sample as positive or negative for a condition, such as lung cancer. A treatment may be an intervention by a medical professional or in the form of providing actionable information to a subject in the form a tangible report (e.g., delivered through a computer system to be displayed to a subject on a graphical user interface, or a paper copy of a report).

An intervention by a medical profession may involve, by way of non-limiting examples, screening, monitoring, or administering therapy. Screening may include various imaging, or diagnostic testing techniques. Screening using imaging may include a low-dose computerized tomography (CT) scan and X-ray. In a non-limiting example, methods and systems of the present disclosure may be used after a lung nodule is identified in an imaging scan. Imaging may be used to screen or monitor a subject after he or she receives classification results. Diagnostic assays may similarly be used to identify a subject as a candidate for use of the methods of systems disclosed in the instant application. Such assays may include but are not limited to sputum cytology, tissue sample biopsy, immunoblot analysis, RNA sequencing or genome sequencing. Monitoring may involve a low-dose computerized tomography (CT) scan, X-ray, sputum cytology, RNA sequencing or genome sequencing.

In the event that a lung condition, such as cancer, is detected using the systems and methods of the instant disclosure, a therapy may be administered to a subject in need thereof. A therapy may involve, for example, the administration of one or more therapeutic agents or a surgical procedure. Non-limiting examples of therapeutic agents include chemotherapeutic agents, monoclonal antibodies, antibody drug conjugates, EGFR inhibitors, and ALK protein binding agents. A surgical procedure may involve, but is not limited to, thoracotomy, lobectomy, thoracoscopy, segmentectomy, wedge resection, or pneumonectomy. Treatment or therapy may include but is not limited to chemotherapy, radiation therapy, immunotherapy, hormone therapy, and pulmonary rehabilitation.

A treatment may be a medical intervention in the form of a report provided to a subject or to a medical professional. A medical professional may act as an intermediary and deliver results directly to a subject. The report may provide information such as the presence or absence of gene fusion(s) and results generated from classifying a sample as positive or negative for a lung condition based in part on assaying nucleic acids from epithelial cells in the subject's respiratory tract, such as lung cancer. The report may provide information regarding potential treatment options, such as potential drugs or clinical trials, based in part on the fusions detected.

By way of illustrative example, if a sample is classified as positive for lung cancer using the systems or methods of the present disclosure, then the subject may receive one or more of chemotherapy, radiation therapy, immunotherapy, hormone therapy, pulmonary rehabilitation, or any combination thereof. In another non-limiting example, if a sample is classified as negative for lung cancer using the systems or methods of the present disclosure, then the subject may be monitored on an on-going basis for potential development of cancerous nodules or lesions.

### Example 1: Identification of Fusions Associated with Lung Cancer

### Data Set

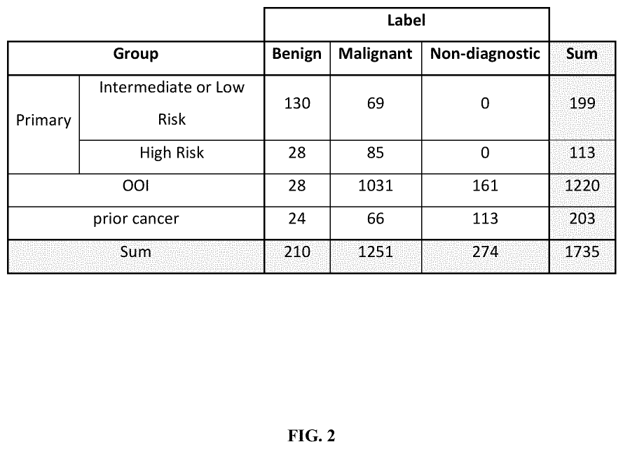

Two types of lung biopsy samples were utilized: bronchial brushings and transbronchial biopsies (TBB). A total of 1,735 bronchial brushing samples were used for fusion detection (FIG. 2). This sample set contains both bronchoscopy positive (mainly in the “OOI” group) and negative samples (mainly in the “Primary” group), and it empowers the classifier algorithm to reduce false discovery in identifying fusions associated with lung cancer by examining the consistency of observed association patterns. 296 TBB samples with negative lung cancer labels were included as a benign patient cohort to filter out fusions that may not be associated with lung cancer. Both types of samples were sequenced and analyzed with the same sequencing pipeline, which analyzes sequence information from both RNA and DNA molecules in a sample. The sequencing pipeline comprises obtaining RNA and DNA from a sample. For RNA, reverse transcription is performed on the RNA molecules to yield cDNA. Sequencing libraries are prepared for both cDNA and DNA from a sample using the Illumina Nextera library prep kit. Prior to sequencing, the libraries are clonally amplified. Amplified molecules corresponding to cDNA and DNA molecules are then sequenced using the Illumina platform, to yield both RNA and DNA sequence data for a sample. FIG. 2 shows a bronchial brushing sample set.

### Classifier Algorithm

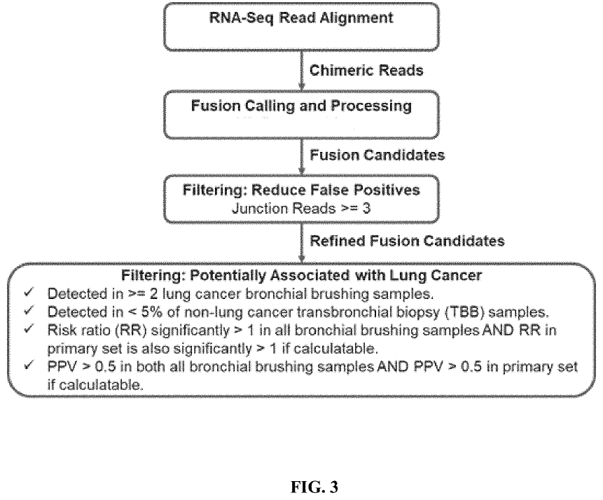

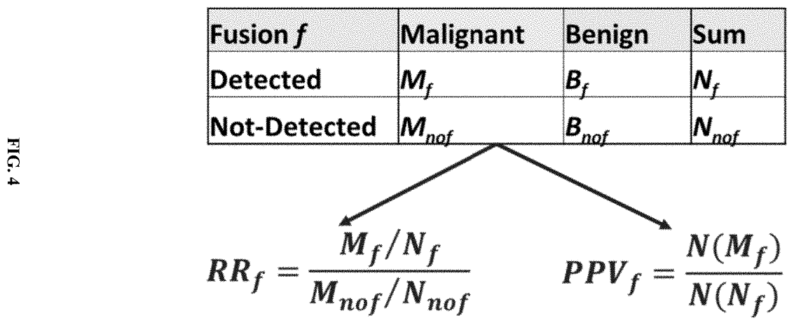

FIG. 3 shows a diagram for detecting lung cancer associated fusions. Fusion detection workflow is depicted in the diagram. The reads from RNA-sequencing were first aligned using a read aligner, and the chimeric reads were passed to a fusion caller. Fusion candidates called were filtered by the number of junction reads to reduce false positives. On the refined fusion candidates, a set of filters were applied to identify fusions that are potentially associated with lung cancer. The filters considered the prevalence of a fusion in both bronchial brushing lung cancer and TBB benign patient cohorts. Risk ratio (RR) was calculated to evaluate the association between fusion detection and cancer label (FIG. 4). FIG. 4 shows a definition of risk ratio and positive predicative value of a fusion f, given contingency table. A fusion is considered to be associated with lung cancer is satisfying: (1) RR>1 (p value <0.05) among all bronchial brushings, and (2) if RR can be calculated in Primary brushing group—meaning a fusion was detected at least once in Primary—RR>1 (p value <0.05) is required. In addition, to quantify the predicative value of a fusion to the lung cancer label, positive predicative value (PPV) of a fusion was considered and PPV>0.5 was required in both all and Primary brushing samples, similarly to RR (FIG. 4).

### Fusion Partner Gene Expression Analysis

There is a common hypothesis that genes involved in cancer related fusions may be differentially expressed compared with samples without fusions. Expression level patterns of the gene partners in each lung cancer associated fusion derived from the workflow were analyzed. Expression level is quantified by variance stabilizing transformation (VST) of RNA-Sequencing read counts for each gene by R package DESeq2. T-test was used to determine if a gene was differentially expressed among samples with or without the fusion of interest.

### Results

Lung Cancer Associated Fusions in Bronchial Brushing Samples

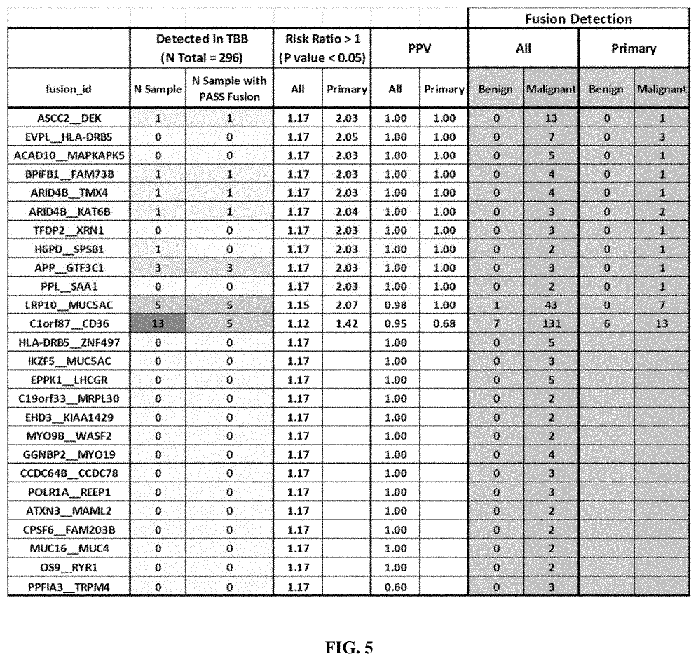

FIG. 5 shows 26 fusions identified by the workflow to be associated with lung cancer. 26 fusions were identified as associated with lung cancer by the workflow. 12 fusions were detected in both all and Primary brushing samples, and except for C1orf87_CD36, the other 11 all have PPV=1.

### Partner Genes in Lung Cancer Associated Fusions are Differentially Expressed.

Differential gene expression analysis was performed on lung cancer associated fusions identified by the workflow. 13 out of 26 fusions contain at least 1 gene partner that is differentially expressed. Given the exploratory nature of this analysis, the p-values were not subjected to multiple hypothesis correction. Gene partners in top 2 most frequent fusions—LRP10_MUC5AC and C1orf87_CD36 may be differentially expressed, compared with samples without fusions.

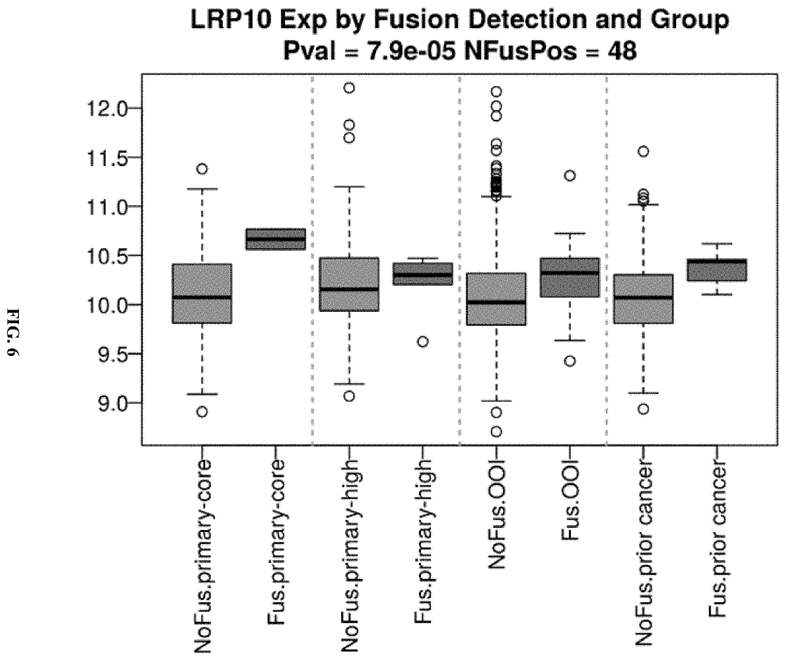

FIGS. 6-9 show expression of the genes in the top 2 most frequent fusions.

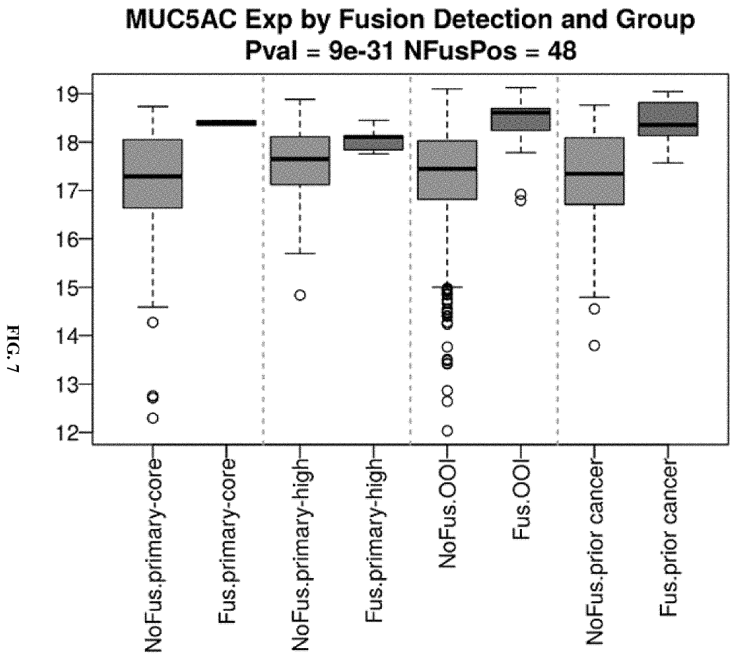

FIGS. 6-7 show both genes in LRP10-MUC5AC fusion showed a higher expression level in samples with this fusion. The samples are divided into those with and without gene fusions and further into primary-core, primary-high, OOI, and prior cancer as shown in FIG. 2. Primary-core are samples with an intermediate or low risk of cancer. Primary-high are lung biopsy samples with a high risk of cancer. Prior cancer samples are those from individuals who had previously been diagnosed with cancer.

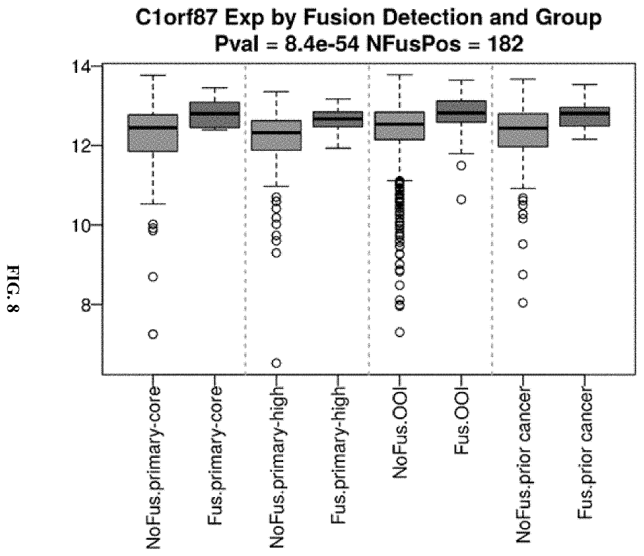

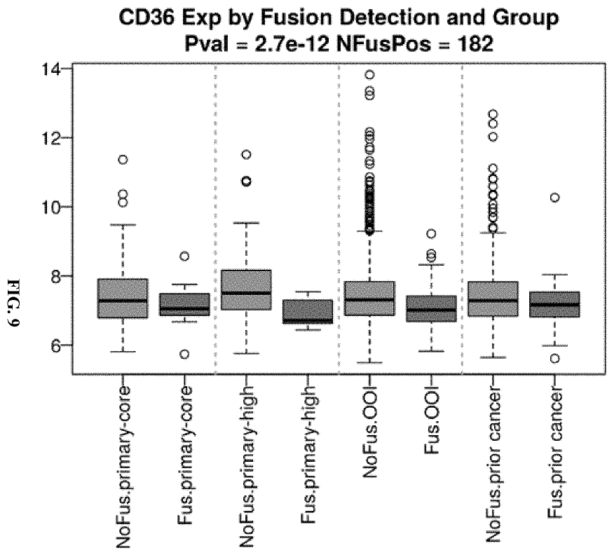

FIGS. 8-9 show C1orf87 shower higher expression level in samples with this fusion, while CD36 showed lower expression level. The samples are divided into those with and without gene fusions and further into primary-core, primary-high, OOI, and prior cancer as can be seen in FIG. 2. Primary-core are samples with an intermediate or low risk of cancer. Primary-high are lung biopsy samples with a high risk of cancer. Prior cancer samples are those from individuals who had previously been diagnosed with cancer.

### Example 2: Status of Whether a Subject is Currently Using an Inhaled Medication

A subgroup of samples was created comprising 162 samples from patients with a low to intermediate pre-test risk of lung disease based on the presence or absence of a pulmonary nodule, as detected by a chest computed tomography (CT) scan, and smoking history. The sample data was obtained from Airway Epithelium Gene Expression in the Diagnosis of Lung Cancer (AEGIS) and the Registry. Out of the 162 samples, 75 were from AEGIS and 87 were from the Registry. Regarding self-reported smoking status, out of the 162 samples 114 were former smokers and 48 were current smokers. Regarding the timing of the sample collection, out of the 162 samples 93 were collected prior to a clinical sample, 57 were collected after a clinical sample, and 12 were missing data with regard to when they were collected. Regarding whether the subjects self-reported using an inhaled medication, out of the 162 samples, 87 said no, 64 said yes, and 11 were missing a response. A Fisher value of p=0.02 was calculated for the relationship between COPD and Inhaled medication, indicating a close association between inhaled medication and COPD. Regarding COPD status, the following information was obtained for the 162 samples:

TABLE 1
COPD Status v. Inhaled Medication

| Primary low- | Using | Not using | |
|---|---|---|---|
| intermediate | inhaled | inhaled | |
| (n = 162) | med. | med. | Unknown/NA |
| COPD | 51 | 7 | 1 |
| Not COPD | 9 | 6 | 1 |
| Missing | 27 | 51 | 9 |

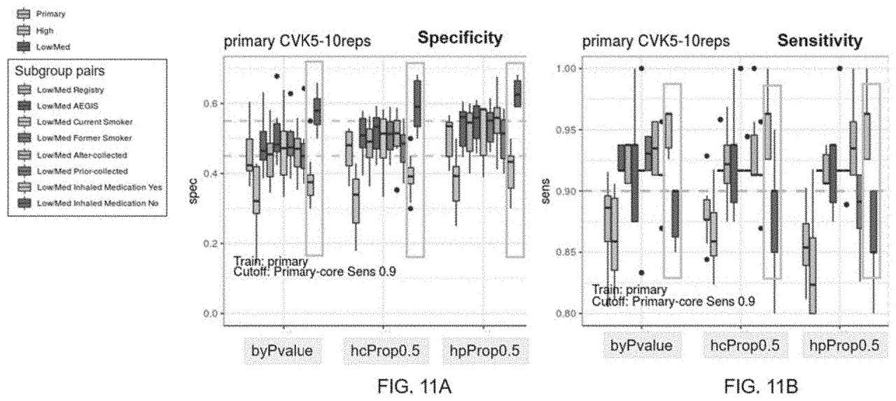

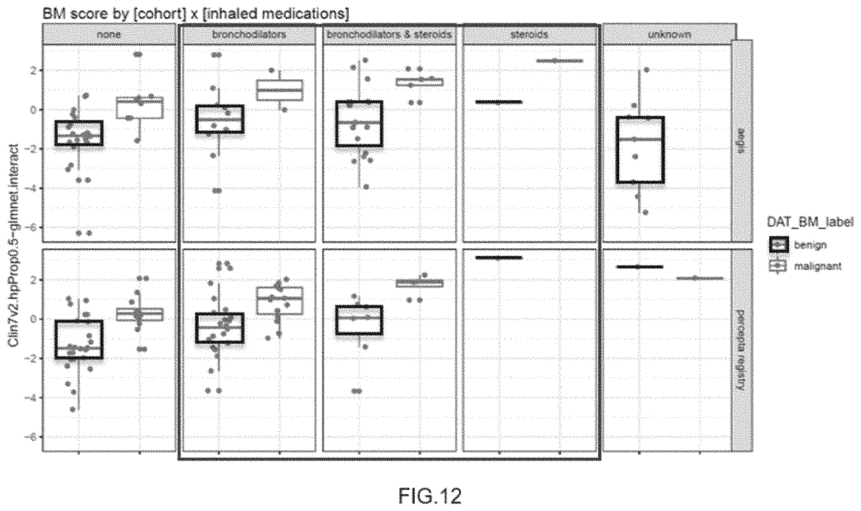

The classifier was applied to the samples (Clin7.v2) and it was found that a shift in specificity outside of the performance threshold and a shift in sensitivity below the performance threshold was seen for samples identified as having used an inhaled medication as can be seen in FIG. 11A and FIG. 11B. An upward score shift was observed for both benign and malignant samples associated with exposure to bronchodilators, steroids, or both as can be seen in FIG. 12. In order to increase the specificity and sensitivity of the algorithm to samples associated with use of an inhaled medication, two approaches were made. The first approach was to add a covariate to the classifier defined as “currently using inhaled medication.” The second approach was to apply a score shift to patients using inhaled medication.

First Approach: Covariate “Currently Using Inhaled Medication”:

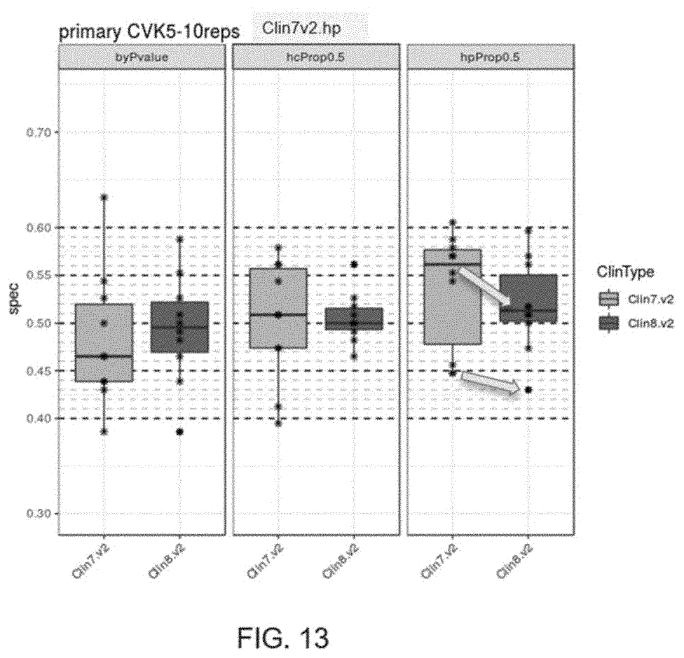

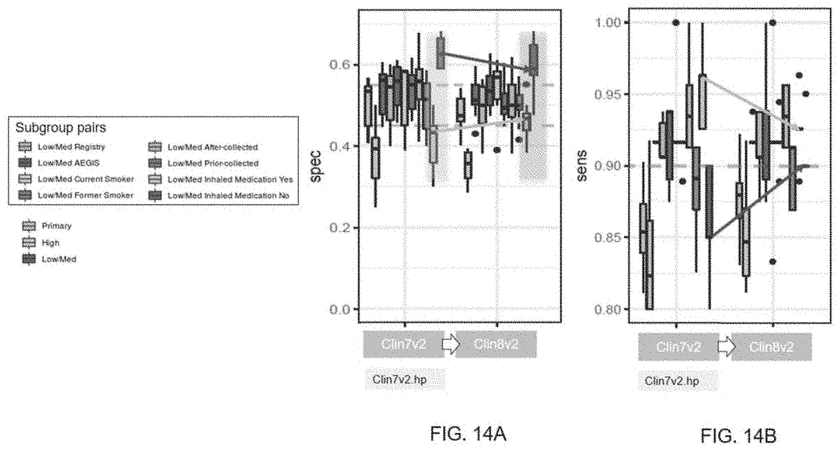

The classifier, as described above, was modified to include whether the patient was currently using inhaled medication (Clin 8.v2) by including a covariate comprising weighted expression levels of genes associated with exposure to inhaled medications. The standard deviation of the specificity dropped from 0.06 to 0.05 and the median specificity dropped from 0.56 to 0.51 as can be seen in FIG. 13. However, as can be seen in FIG. 14A and FIG. 14B the specificity and sensitivity gap between those samples associated with an inhaled medication (bottom right) and those not associated with an inhaled medication (top right) was reduced.

Second Approach: Apply a Score-Shift

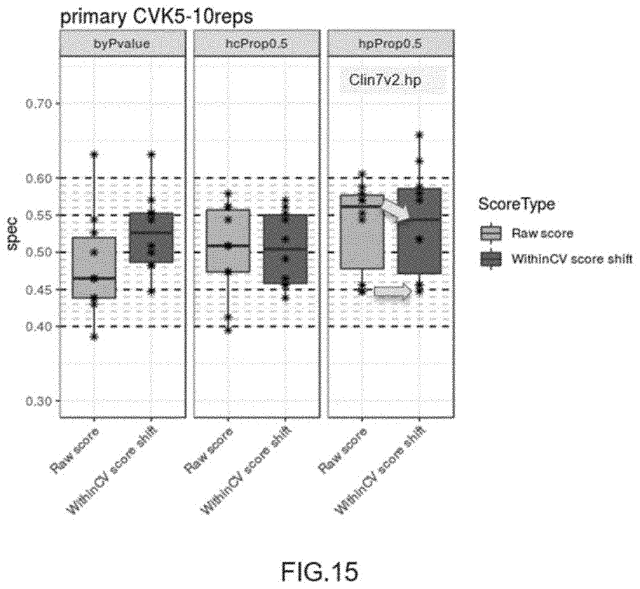

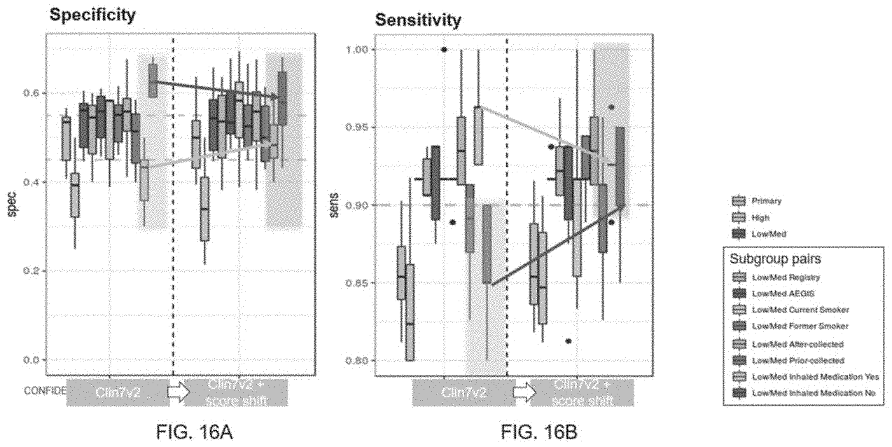

A median score shift was computed within the training set for samples associated with an inhaled medication. The score shift was applied to samples associated with inhaled medication in a test set (Clin7v2+score shift). A minor drop in median specification from 0.56 to 0.54 was observed and the standard deviation of the specificity increased from 0.06 to 0.07 as can be seen in FIG. 15. As can be seen in FIGS. 16A and 16B, the specificity increased to above 0.45 and the sensitivity increased to above 0.9.

### Example 3: Performance of the Genomic Sequencing Classifier on Training Sets

As described in Example 2, samples from AEGIS and the Registry were classified as being associated with inhaled medication or having no association/an unknown association as well as classified by whether the sample is from a patient with COPD. The samples were used to train a classifier (Clin6v2Ens).

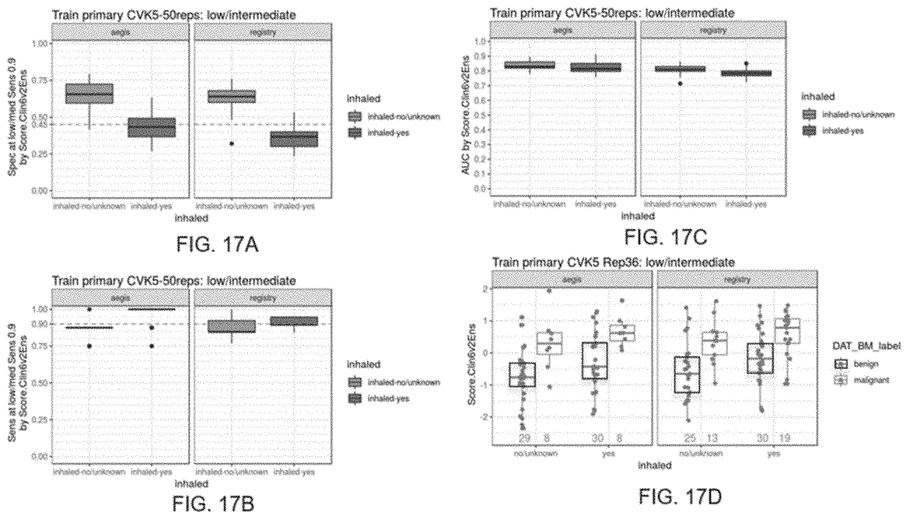

Cohort×Inhaled Medication: As can be seen in FIG. 17A the AEGIS and Registry samples had a similar specificity at low/med sensitivity of 0.9 for samples not associated with inhaled medication. However, both AEGIS and Registry samples failed to meet the specificity threshold for samples associated with inhaled medications, with Registry samples having a lower specificity than AEGIS samples. As can be seen in FIG. 17B the AEGIS and Registry samples had a similar sensitivity, with the samples not associated with inhaled medication having a sensitivity outside of the threshold for both AEGIS and Registry samples. As can be seen in FIG. 17C the AUC for AEGIS samples is slightly higher than the AUC for Registry samples. FIG. 17D shows the scores given for each sample, from each registry according to classification, registry source, and association with inhaled medication.

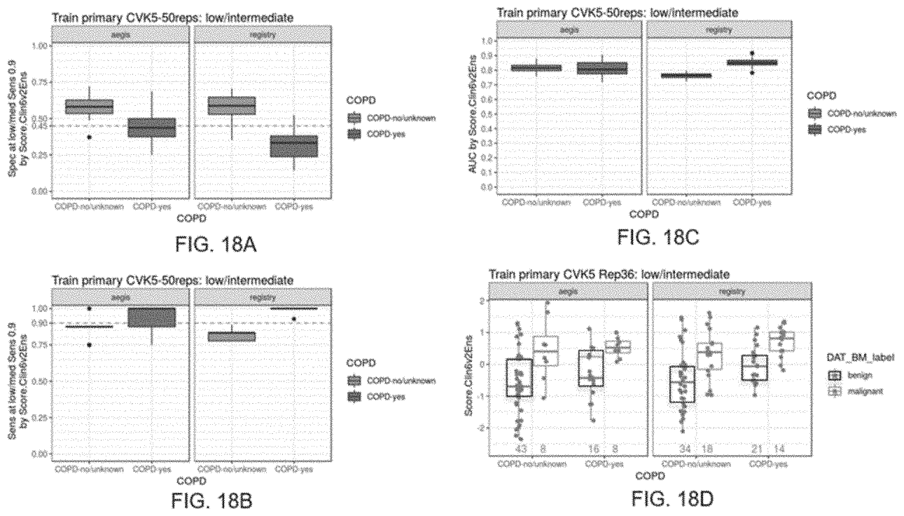

Cohort×COPD: As can be seen in FIG. 18A the AEGIS and Registry samples had a similar specificity at low/med sensitivity of 0.9 for samples not associated with inhaled medication. However, both AEGIS and Registry samples failed to meet the specificity threshold for samples associated with COPD, with Registry samples having a lower specificity than AEGIS samples. As can be seen in FIG. 18B the AEGIS and Registry samples had a similar sensitivity, with the samples not associated with COPD having a sensitivity outside of the threshold for both AEGIS and Registry samples. As can be seen in FIG. 18C the AUC for Registry samples associated with COPD is slightly higher than the AUC for Registry samples. FIG. 18D shows the scores given for each sample, from each registry according to classification, registry source, and association with COPD.

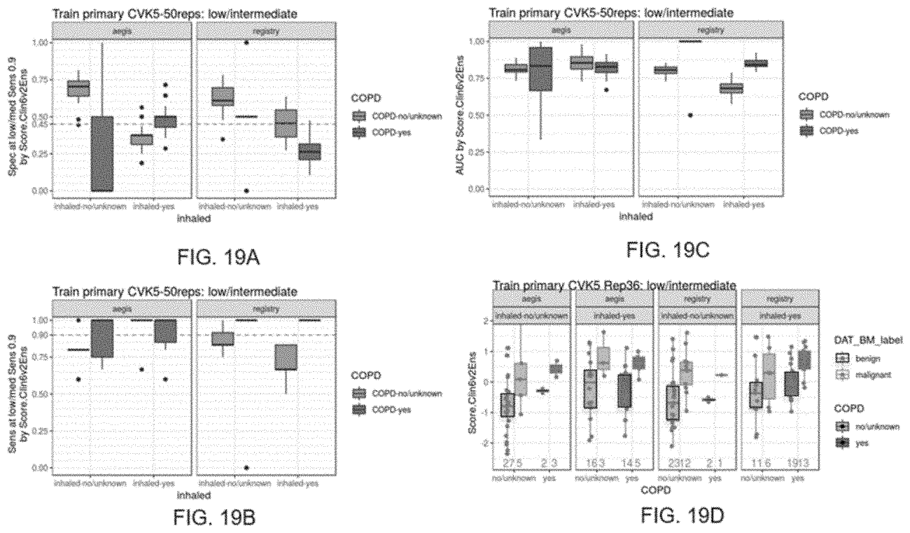

Cohort×Inhaled Medication×COPD: As can be seen in FIG. 19A the AEGIS and Registry samples had a similar specificity at low/med sensitivity of 0.9 for samples not associated with inhaled medication or COPD. However, only AEGIS registry samples failed to meet the threshold for samples associated with inhaled medications but not with COPD. Whereas AEGIS samples associated with COPD but not associated with an inhaled medication showed a large standard deviation and failed to meet the specificity threshold, Registry associated with COPD but not associated with an inhaled medication surpassed the specificity threshold. However, Registry samples associated with COPD and inhaled medications failed to meet the specificity threshold, whereas AEGIS samples associated with COPD and inhaled medications met the threshold. As can be seen in FIG. 19B the AEGIS and Registry samples had a similar sensitivity, with the samples not associated with inhaled medication having a sensitivity outside of the threshold for both AEGIS and Registry samples. As can be seen in FIG. 19C the AUC for AEGIS samples is slightly higher than the AUC for Registry samples. FIG. 19D shows the scores given for each sample, from each registry according to classification, registry source, and association with inhaled medication.

### Example 4: Genomic Sequencing Classifier on Validation for Inhaled Medication

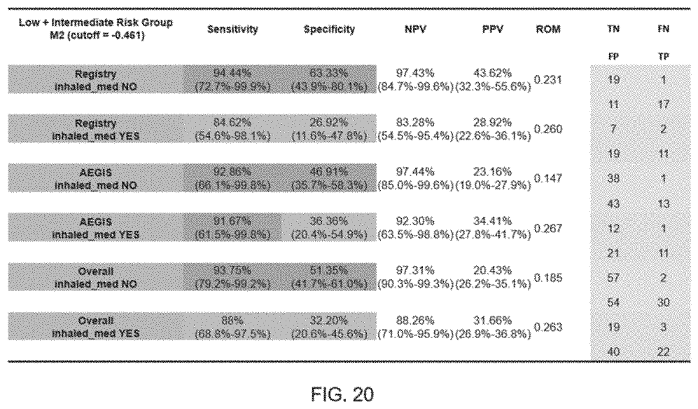

The performance of the classifier was tested by validating Registry samples and AEGIS Samples. FIG. 20 shows a table of the data, naming which registry the sample came from, whether it was associated with an inhaled medication, the calculated sensitivity of the classifier with the data, the calculated specificity of the classifier with the data, the negative predictive value (NPV) of the classifier, the positive predictive value (PPV) of the classifier, the Risk of Malignancy (ROM) and the number of True Negatives (TN), True Positives (TP), False Positives (FP) and False Negatives (FN).

### Example 5: Status of Whether a Sample was Taken Before or after a Clinical Sample

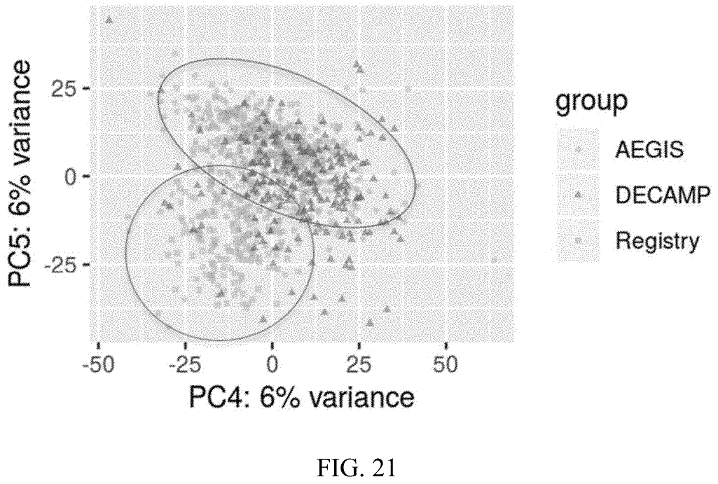

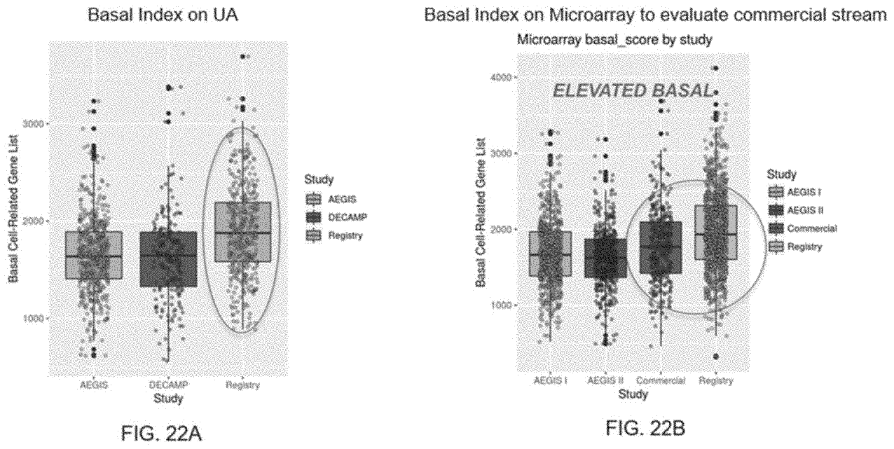

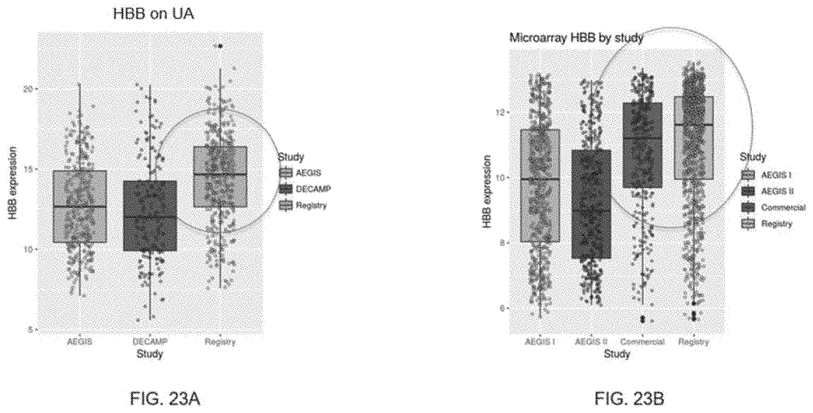

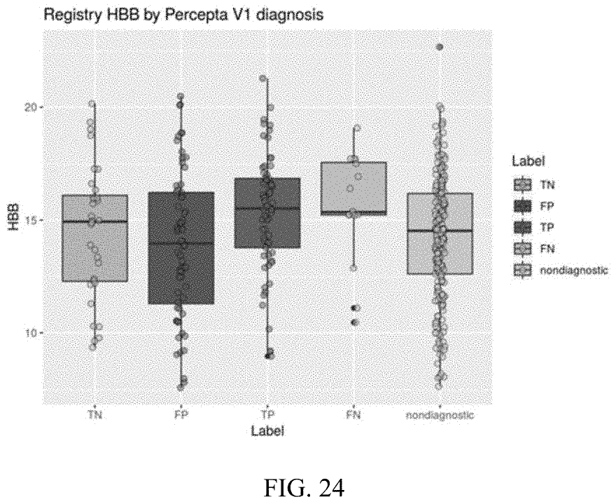

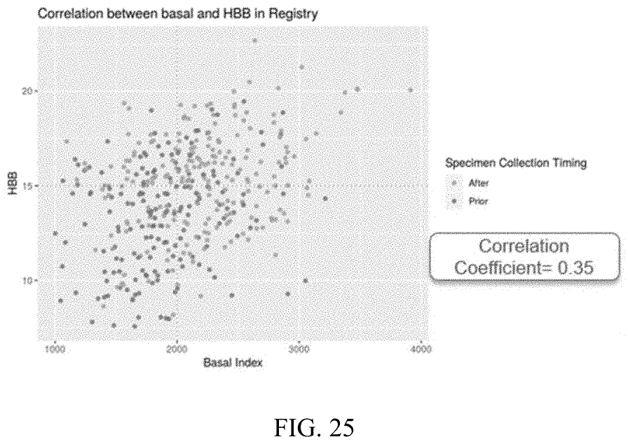

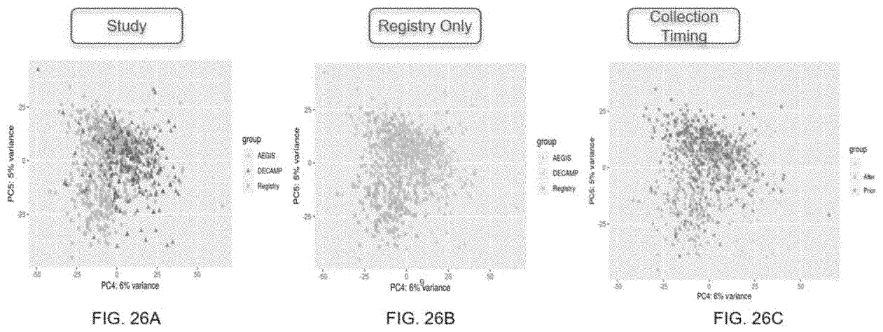

As can be seen in FIG. 21, it was observed that the gene sequencing data between the Registry and two other registries (AEGIS and DECAMP) did not cover the same genomic space. As can be seen in FIG. 22A-22B it was observed that samples from the Registry and the Commercial registry, acquired by both a unified assay (UA, a whole transcriptome platform) and microarray, had an elevated Basal Index as compared to samples from the AEGIS and DECAMP registries. The Basal Index is a composite signal from genes that are present in basal cells. As can be seen in FIG. 23A-23B it was also observed that samples from the Registry and the Commercial registry, acquired by both a unified assay (UA) and microarray, had an elevated expression level of HBB (beta-globulin) as compared to samples from the AEGIS and DECAMP registries. HBB expression level is a marker that can be used to determine the level of blood contamination in a sample. As can be seen in FIG. 24, it was found that False Negative samples from the Registry had a higher level of HBB expression/blood contamination than True Negatives, False Positives, or True Positives. As can be seen in FIG. 25, the time of collection is differentially correlated with basal index expression and HBB expression. “After” collection time samples are associated with elevated HBB and Basal Index. Both basal index and blood content tended to be higher in samples collected after collecting clinical samples. As can be seen in FIG. 26A-26C, the unique expression pattern found in Registry samples can be explained by the fact that the samples tend to be collected after clinical samples. As can be seen in FIG. 26C, samples taken prior to a clinical sample are in the same genomic space as AEGIS and DECAMP registry samples.

Next, the proportion of Registry samples taken before and after a clinical sample were analyzed, as can be seen in Table 2.

TABLE 2
Tumor Subtype v. Collection Timing on Registry Adjudicated Training Set

| | | | | Malignant Only: Cancer subtypes (subset of Total Malignant) | | | | | | |
|---|---|---|---|---|---|---|---|---|---|---|
| | | | | | | | | Malignant | | |
| Collection | Total | Total | Total | Malignant | Malignant | Malignant | Malignant | Mixed Cell | Malignant | |
| timing | Benign | Nondiagnostic | Malignant: | sclc | Squamous | Adeno | Carcinoid | Type | Other | Uncertain |
| Prior | 31 | 29 | 18 | 1 | 5 | 7 | 0 | 1 | 3 | 1 |
| After | 36 | 38 | 38 | 2 | 7 | 18 | 2 | 0 | 2 | 7 |

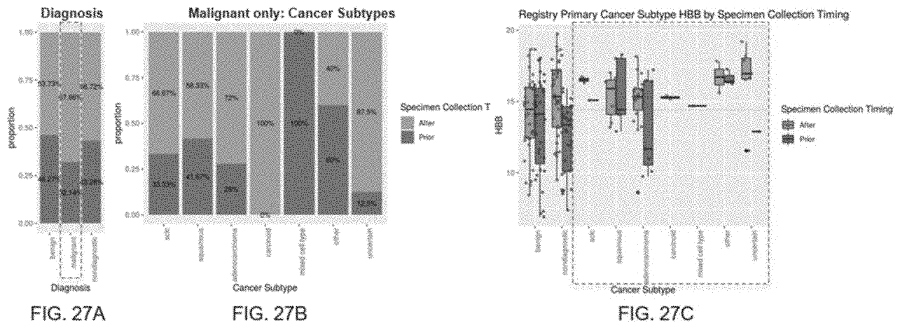

As can be seen in FIG. 27A and FIG. 27B, the samples classified as malignant comprised a higher proportion of samples taken after a clinical sample than samples classified as benign or nondiagnostic. As can be seen in FIG. 27C the standard deviation of samples taken after a clinical sample decreased as compared to the standard deviation of samples taken before a clinical sample.

### Example 6: Genomic Sequencing Classifier on Validation for Timing of Sample Before or after Clinical Sample

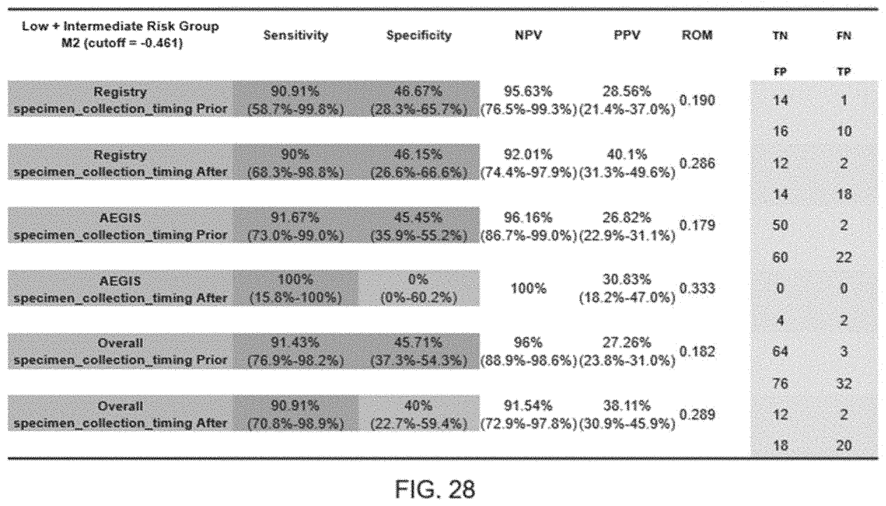

The performance of the classifier was tested by validating Registry samples and AEGIS Samples. FIG. 28 shows a table of the data, naming which registry the sample came from, whether it was associated with an inhaled medication, the calculated sensitivity of the classifier with the data, the calculated specificity of the classifier with the data, the negative predictive value (NPV) of the classifier, the positive predictive value (PPV) of the classifier, the Risk of Malignancy (ROM) and the number of True Negatives (TN), True Positives (TP), False Positives (FP) and False Negatives (FN).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-78. (canceled)

79. A method for processing or analyzing a sample of epithelial tissue from a respiratory tract of a subject, comprising:
(a) providing said sample of epithelial tissue from said respiratory tract of said subject, wherein said sample comprises gene expression products;
(b) assaying said gene expression products of said sample by sequencing, sequence hybridization, array hybridization, or nucleic acid amplification, to yield data;
(c) in a programmed computer, using said data to determine a presence or absence of one or more gene fusions; and
(d) electronically outputting a report that identifies a classification of said sample of epithelial tissue from said respiratory tract of said subject as positive or negative for said lung cancer.

80. The method of claim 79, wherein said sample is a bronchial brushing sample.

81. The method of claim 79, wherein said subject has lung nodules that are inconclusive for lung cancer, as determined by computed tomography scan or bronchoscopy.

82. The method of claim 79, wherein said sample is inconclusive for lung cancer.

83. The method of claim 79, wherein said sample comprises a bronchial epithelial tissue, a nasal epithelial tissue, a lung epithelial tissue, or any combination thereof.

84. The method of claim 79, wherein said sample comprises epithelial tissue obtained along an airway of said subject.

85. The method of claim 79, wherein said gene expression products are ribonucleic acid.

86. The method of claim 79, wherein said sample comprises deoxyribonucleic acid.

87. The method of claim 86, wherein nucleic acid amplification comprises contacting at least one target sequence within said gene expression products with a nucleic acid probe under conditions wherein the probe forms hybridization complexes with said at least one target sequence, wherein said probe comprises the target specific sequence and an adapter sequence that is unique to said gene expression products.

88. The method of claim 79, wherein (c) comprises using a trained algorithm that uses said data to determine said presence or absence of said one or more gene fusions, wherein said trained algorithm is trained by a training data set.

89. The method of claim 88, wherein said training data set comprises data from samples benign for a lung condition and samples malignant for said lung condition.

90. The method of claim 88, wherein said trained algorithm comprises a covariate.

91. The method of claim 90, wherein said covariate is a self-reported characteristic.

92. The method of claim 91, wherein said self-reported characteristic is exposure to an inhaled medication and said covariate is a weight applied to gene expression data of genes associated with exposure to an inhaled medication.

93. The method of claim 88, wherein said training data set comprises samples obtained from subjects using inhaled medication.

94. The method of claim 79, wherein determining further comprises using a trained algorithm wherein said trained algorithm comprises a first filter and a second filter wherein the first filter identifies gene fusion candidates and the second filter identifies refined gene fusion candidates to generate said classification.

95. The method of claim 94, further comprising said first filter filtering said data by the number of junction reads.

96. The method of claim 95, wherein said number of junction reads is greater than three.

97. The method of claim 95, wherein said number of junction reads is equal to three.

98. The method of claim 94, further comprising said second filter identifying refined gene fusion candidates based off scoring in the following:
(i) a prevalence value of a gene fusion in both bronchial brushing lung cancer and TBB benign patient cohorts;
(ii) a risk ratio (RR); and
(iii) a positive predicative value (PPV).
